(19) United States
(12) Patent Application Publication
(10) Pub. No.: US 2021/0299111 A1
(43) Pub. Date: Sep. 30, 2021

(54) EIF4A INHIBITOR COMBINATIONS

(71) Applicant: eFFECTOR Therapeutics Inc., San Diego, CA (US)

(72) Inventors: Peggy A. Thompson, San Diego, CA (US); Kevin R. Webster, San Diego, CA (US)

(21) Appl. No.: 17/210,062

(22) Filed: Mar. 23, 2021

Related U.S. Application Data
(60) Provisional application No. 62/993,889, filed on Mar. 24, 2020.

(51) Int. Cl. A61K 31/4355; A61K 9/00; A61K 45/06; A61P 35/04

(52) U.S. Cl. CPC A61K 31/4355; A61K 9/0019; A61P 35/04; A61K 45/06; A61K 9/0053

(57) ABSTRACT

The present disclosure relates to methods for ameliorating or treating an eIF4A dependent condition or disease in a subject in need thereof. The methods of the disclosure comprise administering to the subject a therapeutically effective amount of at least one eukaryotic translation initiation factor 4A (eIF4A) inhibitor and a therapeutically effective amount of at least one cyclin-dependent kinase (CDK) inhibitor.

### CROSS REFERENCE TO RELATED APPLICATIONS

This application claims priority to U.S. Provisional Patent Application No. 62/993,889, filed Mar. 24, 2020, the disclosure of which is incorporated by reference herein in its entirety, including any drawings and sequence listing.

### BACKGROUND

Dysregulation of the complex regulatory network that controls cell cycle progression is a hallmark of cancer. A major axis of dysregulation is the gateway to cell cycle entry. Cyclin-dependent kinase 4 (CDK4) and Cyclin-dependent kinase 6 (CDK6) are part of the CDK family of serine/threonine kinases that control the transition between the G1 and S phases of the cell cycle. The S phase is the period during which the cell synthesizes new DNA and prepares itself to divide during the process of mitosis. A major target of CDK4 and CDK6 during cell-cycle progression is the retinoblastoma protein (Rb). Rb restricts progression from G₁ phase into S phase by binding and suppressing E2F transcription factors. Regulation of CDK4/6 activity is key to the deactivation of the Rb protein. CDK4 and CDK6 become active when CDK4/6 form heterodimers with D-type cyclins, which are upregulated and post-translationally modified in response to mitogenic signals. Upon activation, CDK4/6 phosphorylate Rb, thereby inactivating the growth-suppressive properties of Rb, which then results in aberrant cell proliferation. This dysregulation of the CDK4/6 activities is a feature of many tumor types. It is therefore unsurprising that CDK4/6 are recognized as key targets for therapeutic intervention. Research has focused on small-molecule inhibition of CDK4/6 function and such CDK4/6 inhibitors have been designed, developed and trialed in the clinic with increasing success over the last few years.

CDK4/6 inhibitors are a class of pharmacological agents used to target dysregulated CDK4/6 activities in malignant cells. The CDK4/6 inhibitors “turn off” these kinases, which results in dephosphorylation of Rb and block of cell-cycle progression in mid-G1. This causes cell-cycle arrest and prevents the proliferation of cancer cells. However, the clinical utility of CDK4/6 inhibitor drugs has been limited by drug resistance—20% of patients do not respond to CDK4/6 inhibitors, and of those initially responding, half develop drug resistance within 25 months.

Accordingly, while advances have been made in this field, there remains a need for improved approaches to treating cancers that are characterized by dysregulated CDK4/6 activation. Presently disclosed embodiments address this need and provide other related advantages.

### BRIEF SUMMARY

In certain embodiments, the present disclosure provides a method for ameliorating or treating an eIF4A dependent condition in a subject in need thereof comprising administering to the subject a therapeutically effective amount of at least one eukaryotic translation initiation factor 4A (eIF4A) inhibitor and a therapeutically effective amount of at least one cyclin-dependent kinase (CDK) inhibitor, wherein the at least one eIF4A inhibitor comprises a compound in accordance with Formula I:

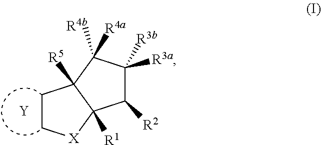

or stereoisomers, tautomers or pharmaceutically acceptable salts thereof,

wherein:

X is CR⁶R⁷, O, S, NH, N(C₁-C₈)alkyl, C(O), C═CR⁶R⁷, N(CO)R⁸, S(O) or S(O)₂;

Y is a 5-membered heteroaryl or a 6-membered aryl or heteroaryl;

R¹ and R² independently are aryl, heterocyclyl, heteroaryl or cycloalkyl;

R^{3a}, R^{3b}, R^{4a} and R^{4b} independently are H, halogen, CN, C₁-C₈(alkyl), (C₁-C₈)haloalkyl, C₂-C₈(alkenyl), (C₂-C₈)alkynyl, OR⁹, NHR⁹, NR⁹R⁹, [(C₁-C₈)alkylene]OR⁹, [(C₁-C₈)alkylene]NHR⁹, [(C₁-C₈)alkylene]NR⁹R⁹, C(O)R⁸, C(O)NHR⁹, C(O)NR⁹R⁹, C(O)[(C₁-C₈)alkylene]NHR⁹, C(O)[(C₁-C₈)alkylene]NR⁹R⁹, CO₂R⁹, C(S)NHR⁹, C(S)NR⁹R⁹, SR⁹, S(O)R⁹, SO₂R⁹, SO₂NHR⁹, SO₂NR⁹R⁹, NH(CO)R⁸, NR⁹(CO)R⁸, NH(CO)NHR⁹, NH(CO)NR⁹R⁹, NR⁹(CO)NHR⁹, NR⁹(CO)NR⁹R⁹, P(O)(OH)(OR⁹), P(O)(OR⁹)(OR⁹), aryl, heteroaryl, cycloalkyl or heterocyclyl;

R^{3a} and R^{3b}, and R^{4a} and R^{4b} independently combine to form oxo or alkenyl, or a cycloalkyl or heterocyclyl ring; or

R^{3a} and R^{4a}, R^{3b} and R^{4b} or R^{4a} and R⁵ together with the carbon atom to which they are attached form a cycloalkyl or heterocyclyl ring; or

R² and R^{3a} together with the carbon atom to which they are attached form a bicyclic ring system;

R⁵ is H, halogen, OH, CN, N3, SR⁹, (C₁-C₈)alkyl, (C₁-C₈)haloalkyl, O(C₁-C₈)alkyl, O(C₁-C₈)haloalkyl, (C₂-C₈)alkynyl, NHC(O)(C₁-C₈)alkyl or heteroaryl;

R⁶ and R⁷ independently are H, CN, halogen, OR⁹, SR⁹, (C₁-C₈)alkyl, NH(R⁹) or NR⁹R⁹;

R⁸ is H, (C₁-C₈)alkyl, (C₁-C₈)haloalkyl, O(C₁-C₈)alkyl, O(C₁-C₈)haloalkyl, cycloalkyl, O(cycloalkyl), heterocyclyl, O(heterocyclyl), aryl, O(aryl), heteroaryl or O(heteroaryl);

R⁹ is H, (C₁-C₈)alkyl, (C₁-C₈)haloalkyl, cycloalkyl, heterocyclyl, [(C₁-C₈)alkylene] heterocyclyl, aryl, [(C₁-C₈)alkylene] aryl or heteroaryl;

wherein the two R⁹'s together with the nitrogen atom to which they are attached of NR⁹R⁹, [(C₁-C₈)alkylene]NR⁹R⁹, C(O)NR⁹R⁹, C(O)[(C₁-C₈)alkylene]NR⁹R⁹, C(S)NR⁹R⁹, SO₂NR⁹R⁹, NH(CO)NR⁹R⁹ or NR⁹(CO)NR⁹R⁹, optionally form a heterocyclyl ring;

wherein any alkyl, alkenyl, cycloalkyl, heterocyclyl, heteroaryl or aryl is optionally substituted with 1, 2, or 3 groups selected from OH, CN, SH, SO₂NH₂, SO₂(C₁-C₄)alkyl, SO₂NH(C₁-C₄)alkyl, halogen, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, NH₂—C(O)-alkylene, NH(Me)-C(O)-alkylene, CH₂—C(O)-lower alkyl, C(O)-lower alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; or

wherein any alkyl, cycloalkyl or heterocyclyl is optionally substituted with oxo;

“m” and “p” are 1, 2, 3, 4, 5 or 6; and

wherein when Y is a 6-membered aryl then X is not O.

In some embodiments, the at least one CDK inhibitor is a CDK4/6 inhibitor. In specific aspects, the CDK4/6 inhibitor is selected from the group consisting of palbociclib, ribociclib, abemaciclib, trilaciclib, flavopiridol (alvocidib), G1T28-1, G1T38, ON123300, AT7519HCl, P276-00, AT7519, JNJ-7706621, SHR6390, PF-06873600, and derivatives thereof.

In other embodiments, the present disclosure provides a method for ameliorating or treating a cancer in a subject in need thereof comprising administering to the subject a therapeutically effective amount of an eIF4A inhibitor and a therapeutically effective amount of a CDK4/6 inhibitor, wherein the eIF4A inhibitor is a compound according to the following formula:

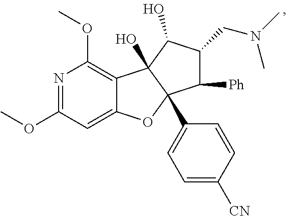

or a stereoisomer, tautomer, or pharmaceutically acceptable salt thereof, and wherein the CDK4/6 inhibitor is selected from the group consisting of palbociclib, ribociclib, and abemaciclib.

The above embodiments and other aspects of this disclosure are readily apparent in the detailed description that follows. Various references are set forth herein which describe in more detail certain background information, procedures and/or compositions, and are each hereby incorporated by reference in their entirety.

### BRIEF DESCRIPTION OF THE DRAWINGS

FIG. 1 shows that the eIF4A inhibitor, eFT226, blocks key cell cycle targets, cyclin D1, CDK4, and phosphorylated retinoblastoma (Rb) protein, in MDA-MB-361 ER⁺ breast cancer cells.

FIG. 2 shows the in vitro effect of a combination of palbociclib and eFT226 on the viability of MDA-MB-361 ER+ breast cancer cells.

FIG. 3 shows the in vivo synergistic effect of a combination treatment using eFT226 and palbociclib on inhibition of tumor cell volume in a mouse xenograft model.

FIG. 4 shows the in vitro synergistic effect of a combination treatment using eFT226 and palbociclib in KRAS mutant cell line SW620.

FIG. 5 shows the in vitro synergistic effect of a combination treatment using eFT226 and palbociclib in KRAS mutant cell line DLD1.

FIG. 6 shows the in vitro synergistic effect of a combination treatment using eFT226 and palbociclib in KRAS mutant cell line CORL23.

### DETAILED DESCRIPTION

In the following description certain specific details are set forth in order to provide a thorough understanding of various embodiments of the invention. However, one skilled in the art will understand that the invention may be practiced without these details. Prior to setting forth this disclosure in more detail, it may be helpful to an understanding thereof to provide definitions of certain terms to be used herein. Additional definitions are set forth throughout this disclosure.

Unless the context requires otherwise, throughout the present specification and claims, the word “comprise” and variations thereof, such as, “comprises” and “comprising” are to be construed in an open, inclusive sense (i.e., as “including, but not limited to”). The term “consisting essentially of” limits the scope of a claim to the specified materials or steps, or to those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

As used herein, and unless noted to the contrary, the following terms and phrases have the meaning noted below.

“Amino” refers to the —NH₂ substituent.

“Aminocarbonyl” refers to the —C(O)NH₂ substituent.

“Carboxyl” refers to the —CO₂H substituent.

“Carbonyl” refers to a —C(O)— or —C(═O)— group. Both notations are used interchangeably within the specification.

“Cyano” refers to the —C≡N substituent.

“Cyanoalkylene” refers to the -(alkylene)CN substituent.

“Acetyl” refers to the —C(O)CH₃ substituent.

“Hydroxy” or “hydroxyl” refers to the —OH substituent.

“Hydroxyalkylene” refers to the -(alkylene)OH substituent.

“Oxo” refers to a ═O substituent.

“Thio” or “thiol” refer to a —SH substituent.

“Alkyl” refers to a saturated, straight or branched hydrocarbon chain radical consisting solely of carbon and hydrogen atoms, having from one to twelve carbon atoms (C₁-C₁₂, alkyl), from one to eight carbon atoms (C₁-C₈ alkyl) or from one to six carbon atoms (C₁-C₆ alkyl), and which is attached to the rest of the molecule by a single bond. Exemplary alkyl groups include methyl, ethyl, n-propyl, 1-methylethyl (iso-propyl), n-butyl, n-pentyl, 1,1-dimethylethyl (t-butyl), 3-methylhexyl, 2-methylhexyl, and the like.

“Lower alkyl” has the same meaning as alkyl defined above but having from one to four carbon atoms (C₁-C₄ alkyl).

“Alkenyl” refers to an unsaturated alkyl group having at least one double bond and from two to twelve carbon atoms (C₂-C₁₂ alkenyl), from two to eight carbon atoms (C₂-C₈ alkenyl) or from two to six carbon atoms (C₂-C₆ alkenyl), and which is attached to the rest of the molecule by a single bond, e.g., ethenyl, propenyl, butenyl, pentenyl, hexenyl, and the like.

“Alkynyl” refers to an unsaturated alkyl group having at least one triple bond and from two to twelve carbon atoms (C₂-C₁₂ alkynyl), from two to ten carbon atoms (C₂-C₁₀ alkynyl) from two to eight carbon atoms (C₂-C₈ alkynyl) or from two to six carbon atoms (C₂-C₆ alkynyl), and which is attached to the rest of the molecule by a single bond, e.g., ethynyl, propynyl, butynyl, pentynyl, hexynyl, and the like.

“Alkylene” or “alkylene chain” refers to a straight or branched divalent hydrocarbon (alkyl) chain linking the rest of the molecule to a radical group, consisting solely of carbon and hydrogen, respectively. Alkylenes can have from one to twelve carbon atoms, e.g., methylene, ethylene, propylene, n-butylene, and the like. The alkylene chain is attached to the rest of the molecule through a single or double bond. The points of attachment of the alkylene chain to the rest of the molecule can be through one carbon or any two carbons within the chain. “Optionally substituted alkylene” refers to alkylene or substituted alkylene.

“Alkenylene” refers to divalent alkene. Examples of alkenylene include without limitation, ethenylene (—CH═CH—) and all stereoisomeric and conformational isomeric forms thereof “Substituted alkenylene” refers to divalent substituted alkene. “Optionally substituted alkenylene” refers to alkenylene or substituted alkenylene.

“Alkynylene” refers to divalent alkyne. Examples of alkynylene include without limitation, ethynylene, propynylene. “Substituted alkynylene” refers to divalent substituted alkyne.

“Alkoxy” refers to a radical of the formula —ORₐ where Rₐ is an alkyl having the indicated number of carbon atoms as defined above. Examples of alkoxy groups include without limitation —O-methyl (methoxy), —O-ethyl (ethoxy), —O-propyl (propoxy), —O-isopropyl (iso propoxy) and the like.

“Acyl” refers to a radical of the formula —C(O)Rₐ where Rₐ is an alkyl having the indicated number of carbon atoms.

“Alkylaminyl” refers to a radical of the formula —NHRₐ or —NRₐRₐ where each Rₐ is, independently, an alkyl radical having the indicated number of carbon atoms as defined above.

“Cycloalkylaminyl” refers to a radical of the formula —NHRₐ where Rₐ is a cycloalkyl radical as defined herein.

“Alkylcarbonylaminyl” refers to a radical of the formula —NHC(O)Rₐ, where Rₐ is an alkyl radical having the indicated number of carbon atoms as defined herein.

“Cycloalkylcarbonylaminyl” refers to a radical of the formula —NHC(O)Rₐ, where Rₐ is a cycloalkyl radical as defined herein.

“Alkylaminocarbonyl” refers to a radical of the formula —C(O)NHRₐ or —C(O)NRₐRₐ, where each Rₐ is independently, an alkyl radical having the indicated number of carbon atoms as defined herein.

“Cyclolkylaminocarbonyl” refers to a radical of the formula —C(O)NHRₐ, where Rₐ is a cycloalkyl radical as defined herein.

“Aryl” refers to a hydrocarbon ring system radical comprising hydrogen, 6 to 18 carbon atoms and at least one aromatic ring. Exemplary aryls are hydrocarbon ring system radical comprising hydrogen and 6 to 9 carbon atoms and at least one aromatic ring; hydrocarbon ring system radical comprising hydrogen and 9 to 12 carbon atoms and at least one aromatic ring; hydrocarbon ring system radical comprising hydrogen and 12 to 15 carbon atoms and at least one aromatic ring; or hydrocarbon ring system radical comprising hydrogen and 15 to 18 carbon atoms and at least one aromatic ring. For purposes of this invention, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. “Optionally substituted aryl” refers to an aryl group or a substituted aryl group.

“Arylene” denotes divalent aryl, and “substituted arylene” refers to divalent substituted aryl.

“Aralkyl” or “araalkylene” may be used interchangeably and refer to a radical of the formula —R_{b}—R_{c} where R_{b} is an alkylene chain as defined herein and R_{c} is one or more aryl radicals as defined herein, for example, benzyl, diphenylmethyl and the like.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, three to nine carbon atoms, three to eight carbon atoms, three to seven carbon atoms, three to six carbon atoms, three to five carbon atoms, a ring with four carbon atoms, or a ring with three carbon atoms. The cycloalkyl ring may be saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like.

“Cycloalkylalkylene” or “cycloalkylalkyl” may be used interchangeably and refer to a radical of the formula —R_{b}Rₑ, where R_{b} is an alkylene chain as defined herein and Rₑ is a cycloalkyl radical as defined herein. In certain embodiments, R_{b} is further substituted with a cycloalkyl group, such that the cycloalkylalkylene comprises two cycloalkyl moieties. Cyclopropylalkylene and cyclobutylalkylene are exemplary cycloalkylalkylene groups, comprising at least one cyclopropyl or at least one cyclobutyl group, respectively.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo (bromine), chloro (chlorine), fluoro (fluorine), or iodo (iodine).

“Haloalkyl” refers to an alkyl radical having the indicated number of carbon atoms, as defined herein, wherein one or more hydrogen atoms of the alkyl group are substituted with a halogen (halo radicals), as defined above. The halogen atoms can be the same or different. Exemplary haloalkyls are trifluoromethyl, difluoromethyl, trichloromethyl, 2,2,2-trifluoroethyl, 1,2-difluoroethyl, 3-bromo-2-fluoropropyl, 1,2-dibromoethyl, and the like.

“Heterocyclyl,” “heterocycle,” or “heterocyclic ring” refers to a stable 3- to 18-membered saturated or unsaturated radical which consists of two to twelve carbon atoms and from one to six heteroatoms, for example, one to five heteroatoms, one to four heteroatoms, one to three heteroatoms, or one to two heteroatoms selected from the group consisting of nitrogen, oxygen and sulfur. Exemplary heterocycles include without limitation stable 3-15 membered saturated or unsaturated radicals, stable 3-12 membered saturated or unsaturated radicals, stable 3-9 membered saturated or unsaturated radicals, stable 8-membered saturated or unsaturated radicals, stable 7-membered saturated or unsaturated radicals, stable 6-membered saturated or unsaturated radicals, or stable 5-membered saturated or unsaturated radicals.

Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of non-aromatic heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, thietanyl, trithianyl, tetrahydropyranyl, thiomorpholinyl, thiamorpholinyl, 1-oxo-thiomorpholinyl, and 1,1-dioxo-thiomorpholinyl. Heterocyclyls include heteroaryls as defined herein, and examples of aromatic heterocyclyls are listed in the definition of heteroaryls below.

“Heterocyclylalkyl” or “heterocyclylalkylene” refers to a radical of the formula —R_{b}R_{f} where R_{b} is an alkylene chain as defined herein and R_{f} is a heterocyclyl radical as defined above, and if the heterocyclyl is a nitrogen-containing heterocyclyl, the heterocyclyl may be attached to the alkyl radical at the nitrogen atom.

“Heteroaryl” or “heteroarylene” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical may be a stable 5-12 membered ring, a stable 5-10 membered ring, a stable 5-9 membered ring, a stable 5-8 membered ring, a stable 5-7 membered ring, or a stable 6 membered ring that comprises at least 1 heteroatom, at least 2 heteroatoms, at least 3 heteroatoms, at least 4 heteroatoms, at least 5 heteroatoms or at least 6 heteroatoms. Heteroaryls may be a monocyclic, bicyclic, tricyclic or tetracyclic ring system, which may include fused or bridged ring systems; and the nitrogen, 2 carbon or sulfur atoms in the heteroaryl radical may be optionally oxidized; the nitrogen atom may be optionally quaternized. The heteroatom may be a member of an aromatic or non-aromatic ring, provided at least one ring in the heteroaryl is aromatic. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl).

“Heteroarylalkyl” or “heteroarylalkylene” refers to a radical of the formula —R_{b}R_{g} where R_{b} is an alkylene chain as defined above and R_{g} is a heteroaryl radical as defined above.

“Thioalkyl” refers to a radical of the formula —SRₐ where Rₐ is an alkyl radical as defined above containing one to twelve carbon atoms, at least 1-10 carbon atoms, at least 1-8 carbon atoms, at least 1-6 carbon atoms, or at least 1-4 carbon atoms.

“Heterocyclylaminyl” refers to a radical of the formula —NHR_{f} where R_{f} is a heterocyclyl radical as defined above.

“Thione” refers to a ═S group attached to a carbon atom of a saturated or unsaturated (C₃-C₈)cyclic or a (C₁-C₈)acyclic moiety.

“Sulfoxide” refers to a —S(O)— group in which the sulfur atom is covalently attached to two carbon atoms.

“Sulfone” refers to a —S(O)₂— group in which a hexavalent sulfur is attached to each of the two oxygen atoms through double bonds and is further attached to two carbon atoms through single covalent bonds.

The term “oxime” refers to a —C(Rₐ)═N—ORₐ radical where Rₐ is hydrogen, lower alkyl, an alkylene or arylene group as defined above.

The compound of the invention can exist in various isomeric forms, as well as in one or more tautomeric forms, including both single tautomers and mixtures of tautomers. The term “isomer” is intended to encompass all isomeric forms of a compound of this invention, including tautomeric forms of the compound.

Some compounds described here can have asymmetric centers and therefore exist in different enantiomeric and diastereomeric forms. A compound of the invention can be in the form of an optical isomer or a diastereomer. Accordingly, the invention encompasses compounds of the invention and their uses as described herein in the form of their optical isomers, diastereoisomers and mixtures thereof, including a racemic mixture. Optical isomers of the compounds of the invention can be obtained by known techniques such as asymmetric synthesis, chiral chromatography, or via chemical separation of stereoisomers through the employment of optically active resolving agents.

Unless otherwise indicated, “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

In this description, a “pharmaceutically acceptable salt” is a pharmaceutically acceptable, organic or inorganic acid or base salt of a compound of the invention (i.e., the eIF4A inhibitors and the CDK inhibitors disclosed herein). Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

As used herein, the term “derivative” refers to a modification of a compound by chemical or biological means, with or without an enzyme, which modified compound is structurally similar to a parent compound and (actually or theoretically) derivable from that parent compound. Generally, a “derivative” differs from an “analog” in that a parent compound may be the starting material to generate a “derivative,” whereas the parent compound may not necessarily be used as the starting material to generate an “analog.” A derivative may have different chemical, biological or physical properties from the parent compound, such as being more hydrophilic or having altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). Other exemplary derivatizations include glycosylation, alkylation, acylation, acetylation, ubiquitination, esterification, and amidation.

The term “derivative” also refers to all solvates, for example, hydrates or adducts (e.g., adducts with alcohols), active metabolites, and salts of a parent compound. The type of salt depends on the nature of the moieties within the compound. For example, acidic groups, such as carboxylic acid groups, can form alkali metal salts or alkaline earth metal salts (e.g., sodium salts, potassium salts, magnesium salts, calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts with, for example, inorganic acids such as hydrochloric acid, sulfuric acid or phosphoric acid, or with organic carboxylic acids or sulfonic acids such as acetic acid, citric acid, lactic acid, benzoic acid, maleic acid, fumaric acid, tartaric acid, methanesulfonic acid or p-toluenesulfonic acid. Compounds that simultaneously contain a basic group and an acidic group, for example, a carboxyl group in addition to basic nitrogen atoms, can be present as zwitterions. Salts can be obtained by customary methods known to those skilled in the art, for example, by combining a compound with an inorganic or organic acid or base in a solvent or diluent, or from other salts by cation exchange or anion exchange.

As used herein, the term “eIF4A,” also known as “eukaryotic initiation factor-4A,” refers to a member of the “DEAD box” family of ATP-dependent helicases that are characterized by seven highly conserved amino acid motifs implicated in RNA remodeling. eIF4A acts as an RNA dependent ATPase and ATP-dependent RNA helicase to facilitate mRNA binding to the ribosome as part of the eIF4F (eukaryotic initiation factor 4F) complex that recognizes and initiates translation of most cellular mRNAs to synthesize specific proteins. A functional eIF4F complex consisting of eIF4A, eIF4E and eIF4G is involved in translation of mRNAs that contain highly structured 5′-UTRs or an IRES element. In particular, eIF4F recognizes the cap structure at the 5′-end of mRNA through eIF4E, unwinds the secondary structure of the 5′-UTR region through the helicase activity of eIF4A, and binds the 43S complex through interactions between eIF4G and eIF3. See, e.g., Marintchev et al., Cell, 136: 447-460, 2009, and Parsyan et al., Nat. Rev. Mol. Cell Biol. 12:235-245, 2012. eIF4A selectively regulates the translation of a subset of mRNAs. This selectivity is a result of structural elements and sequence recognition motifs found within the 5′-UTR of the mRNA. There are three eIF4A family members: eIF4AI, eIF4AII, and eIF4AIII. In particular embodiments, eIF4A refers to human eIF4A. Overexpression of eIF4A has been associated with poor prognosis in various cancers, including lymphoma, lung cancer, colon cancer, liver cancer, ovarian cancer and breast cancer.

As used herein, the term “eIF4A dependent condition” is a disease or condition in a subject resulting from or characterized by an inactive, partially active, or hyperactive eIF4A. In certain embodiments, the eIF4A dependent condition is a disease of uncontrolled cell growth, proliferation and/or survival, or is a disease of inappropriate cellular inflammatory responses. In certain aspects, the eIF4A dependent condition is a disease of uncontrolled cell growth, proliferation and/or survival. In some aspects, the eIF4A dependent condition is a hyperproliferative disease. In specific aspects, the eIF4A dependent condition is cancer. In certain embodiments, the eIF4A dependent condition is a solid tumor, colorectal cancer, bladder cancer, gastric cancer, thyroid cancer, esophageal cancer, head and neck cancer, brain cancer, malignant glioma, fibrotic diseases, glioblastoma, hepatocellular cancers, thyroid cancer, lung cancer, non-small cell lung cancer, small cell lung cancer, melanoma, multiple melanoma, myeloma, pancreatic cancer, pancreatic carcinoma, renal cell carcinoma, renal cancer, cervical cancer, urothelial cancer, prostate cancer, castration-resistant prostate cancer, ovarian cancer, breast cancer, triple-negative breast cancer, leukemia, acute myeloid leukemia, Hodgkins lymphoma, non-Hodgkins lymphoma, B-cell lymphoma, T-cell lymphoma, hairy cell lymphoma, diffuse large B-cell lymphoma, Burkitt's lymphoma, multiple myeloma, myelodysplastic syndrome, Alzheimer's, Parkinson's, Fragile X Syndrome and autism disorders. In specific embodiments, the eIF4A dependent condition includes, without limitation, hepatocellular cancers, breast cancer, small cell lung cancer and non-small cell lung cancer. In additional embodiments, the eIF4A dependent condition is diffuse large B-cell lymphoma, Burkitt's lymphoma, acute myeloid leukemia, triple-negative breast cancer and colorectal cancer.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

As used herein, the term “hyperproliferative disorder” or “hyperproliferative disease” refers to excessive growth or proliferation as compared to a normal cell or an undiseased cell. Exemplary hyperproliferative disorders include dysplasia, neoplasia, non-contact inhibited or oncogenically transformed cells, tumors, cancers, carcinoma, sarcoma, malignant cells, pre-malignant cells, as well as non-neoplastic or non-malignant hyperproliferative disorders (e.g., adenoma, fibroma, lipoma, leiomyoma, hemangioma, fibrosis, restenosis, or the like). In certain aspects, the hyperproliferative disease is cancer. In certain embodiments, a cancer being treated by the compositions and methods of this disclosure includes carcinoma (epithelial), sarcoma (connective tissue), lymphoma or leukemia (hematopoietic cells), germ cell tumor (pluripotent cells), blastoma (immature “precursor” cells or embryonic tissue), or any combination thereof. These various forms of hyperproliferative disease are known in the art and have established criteria for diagnosis and classification (e.g., Hanahan and Weinberg, Cell 144:646, 2011; Hanahan and Weinberg Cell 100:57, 2000; Cavallo et al., Canc. Immunol. Immunother. 60:319, 2011; Kyrigideis et al., J. Carcinog. 9:3, 2010).

The term “inhibit” or “inhibitor” refers to an alteration, interference, reduction, down regulation, blocking, abrogation or degradation, directly or indirectly, in the expression, amount or activity of a target or signaling pathway relative to (1) a control, endogenous or reference target or pathway, or (2) the absence of a target or pathway, wherein the alteration, interference, reduction, down regulation, blocking, abrogation or degradation is statistically, biologically, or clinically significant.

For example, an “eIF4A inhibitor,” as used herein, refers to an agent or compound that directly interacts with eIF4A, either alone or in a complex (e.g., a ternary complex of an eIF4A inhibitor, an eIF4A and a mRNA) and blocks, inactivates, reduces or minimizes eIF4A activity (e.g., helicase activity or translational effects) by about 1%, 2%, 3%, 4%, 5%, 10%, 15%, 20%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more as compared to untreated eIF4A. In some embodiments, the eIF4A inhibitor reduces activity by promoting degradation of eIF4A. In certain embodiments, an eIF4A inhibitor is a catalytic inhibitor that directly inhibits eIF4A helicase activity. An example of an eIF4A catalytic inhibitor is BPSL1549, a bacterial toxin from Burkholderia pseudomallei that deamidates Gln339 of eIF4A and converts it into a dominant-negative mutant (Cruz-Migoni et al., Science 334:821-824, 2011, which inhibitor is incorporated herein by reference in its entirety). Non-limiting examples of inhibitors include small molecules, antisense molecules, ribozymes, RNAi molecules, or the like.

In further embodiments, an eIF4A inhibitor is a chemical inducer of dimerization. An eIF4A chemical inducer of dimerization causes a non-sequence specific interaction between eIF4A and RNA and stimulate the ATP hydrolysis activity of eIF4A, resulting in sequestering of free eIF4A and depletion of eIF4A from the eIF4F complex. Examples of eIF4A inhibitors that are chemical inducers of dimerization include pateamine A, and analogs, derivatives, or precursors thereof. Examples of pateamine A derivatives have been described in U.S. Pat. No. 7,230,021; PCT Publication WO 2016/161168 (α-amino derivatives that lack the C5-methyl group); and U.S. Pat. No. 7,737,134 (desmethyl, desamino-pateamine A derivatives), each derivative of which is incorporated by reference in its entirety.

In still further embodiments, an eIF4A inhibitor is a site-directed eIF4A inhibitor. A “site-directed eIF4A inhibitor,” as used herein, refers to an agent or compound that interacts with a specific nucleotide sequence of a mRNA molecule, such as a non-coding nucleotide sequence (e.g., located in the 5′-UTR of a target mRNA), and is capable of forming a stable ternary complex comprised of the site-directed eIF4A inhibitor, an eIF4A and a target mRNA. Exemplary site-directed eIF4A inhibitors include silvestrol, rocaglamide compounds, as well as analogs, derivatives, or precursors thereof. Representative silvestrol derivatives and analogs include CR-1-31-B, hydroxamate derivative of silvestrol (Rodrigo et al., J. Med. Chem. 55:558-562, 2012; which compounds are incorporated herein by reference in their entirety); episilvestrol (Hwang et al., J. Org. Chem. 69:3350-3358, 2004; which compound is incorporated herein by reference in its entirety); Compounds 74 and 76 (Liu et al., J. Med. Chem. 55:8859-8878, 2012, which compounds are incorporated herein by reference in their entirety), silvestrol dioxane, episilvesterol dioxane, Flavagline 61, (−)-4′-desmethoxyepisilvestrol, and 1-O-formylaglafoline (FA). Examples of rocaglates and precursors include aglapervirisin A and aglapervirisins B-J (An et al., Scientific Reports, Article No. 20045, 2016). Further examples of naturally silvestrol and rocaglamide derivatives and analogs are described in Pan et al., Nat. Prod. Rep. 31:924-939, 2014; Kim et al., Anticancer Agents Med Chem. 6:319-45, 2006; and U.S. Patent Publication US 2014/0255432, compounds from each of which is incorporated herein by reference in its entirety.

Inhibition of eIF4A may be measured by, for example, decreased rates or amounts of protein translation. For example, in certain embodiments, administration of a therapeutically effective amount of an eIF4A inhibitor may reduce translation of c-Myc, Mcl-1, and/or cyclin D1, in a solid tumor by at least about 1.5-fold (e.g., at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold or more) as compared to an untreated reference solid tumor.

“Cyclin-dependent kinases (CDKs)” are important regulators that control the timing and coordination of the cell cycle. The initial discovery of cyclin-dependent kinases was in the context of the cell cycle where “cyclins” were cyclically degraded and includes CDK1, CDK2, CDK3, CDK4 and CDK6. These CDKs form reversible complexes with their obligate cyclin partners to control transition through key junctures in the cell cycle. For example, CDK4 and the closely related CDK6 are regulators of mammalian mitosis, acting to promote the start of DNA synthesis in preparation for cell division. Upon activation by complexing with D-type cyclins, CDK4/6 phosphorylate and inactivate the retinoblastoma protein (Rb); this uncouples the inhibitory interaction between Rb and E2F transcription factors, which initiate a transcriptional program promoting cell cycle progression. In addition to regulating cell cycle progression, further CDK family members have been identified for the transcriptional machinery (CDK7, CDK8, CDK9, CDK12), DNA damage response (CDK12) and in tissue specific functions (CDK5). Despite these diverse functions, the CDKs are structurally very similar, due to the fact that context-specific cyclins are activated to control each function.

A “cyclin-dependent kinase inhibitor,” as used herein, refers to a class of pharmacological agents or compounds used to target dysregulated cyclin-dependent kinase (CDK) activity in malignant cells. CDK inhibitors selectively interact with one or more CDK proteins and block, inactivate, reduce or minimize the activity of the CDKs by about 1%, 2%, 3%, 4%, 5%, 10%, 15%, 20%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more as compared to untreated CDKs. For example, in certain embodiments, administration of a therapeutically effective amount of a CDK inhibitor may inhibit one or more CDK activities in a subject by at least about 1.5-fold (e.g., at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, or more) as compared to the CDK activities in an untreated subject. Exemplary CDK inhibitors inhibit the expression of MCL-1. Exemplary CDK inhibitors include, but are not limited to, alvocidib, dinaciclib, olomoucine, roscovitine, purvalanol, paullones, palbociclib, thio/oxoflavopiridols, oxindoles, aminothiazoles, benzocarbazoles, pyrimidines and seliciclib.

A “CDK4/6 inhibitor” as used herein, refers to a pharmacological agent or compound that selectively interacts with CDK4 and CDK6 (“CDK4/6”) and blocks, inactivates, reduces or minimizes the activity of CDK4 and CDK6 by about 1%, 2%, 3%, 4%, 5%, 10%, 15%, 20%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more as compared to untreated CDK4/6. For example, in certain embodiments, administration of a therapeutically effective amount of a CDK4/6 inhibitor may inhibit CDK4/6 kinase activity in a subject by at least about 1.5-fold (e.g., at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, or more) as compared to CDK4/6 kinase activity in an untreated subject. Several selective CDK4/6 inhibitors are at various stages of development. All CDK4/6 inhibitor compounds are designed by targeting the ATP-binding domains of these proteins. Currently, three highly selective CDK4/6 inhibitors, palbociclib (PD-0332991), ribociclib (LEE001) and abemaciclib (LY2835219), are FDA-approved for treating estrogen receptor-positive (ER⁺) advanced breast cancers. Other known CDK4/6 inhibitors include, but are not limited to, trilaciclib, flavopiridol (alvocidib), G1T28-1, G1T38, ON123300, AT7519HCl, P276-00, AT7519, JNJ-7706621, SHR6390, pharmaceutically acceptable salts thereof, and derivatives thereof. These inhibitors are highly selective for CDK4/6 over other members of the CDK family.

“Treatment,” “treating” or “ameliorating” refers to medical management of a disease, disorder, or condition of a subject (i.e., patient), which may be therapeutic, prophylactic/preventative, or a combination treatment thereof. A treatment may improve or decrease the severity at least one symptom of a disease, delay worsening or progression of a disease, delay or prevent onset of additional associated diseases. In certain embodiments, such terms refer to minimizing the spread or worsening of the disease resulting from the administration of one or more prophylactic or therapeutic agents to a subject with such a disease. In the context of the present invention the terms “treat,” “treating,” “treatment” and “ameliorating” also refer to:

(i) preventing the disease or condition from occurring in a subject, in particular, when such subject is predisposed to the condition but has not yet been diagnosed as having it;

(ii) inhibiting the disease or condition, i.e., arresting its development;

(iii) relieving the disease or condition, i.e., causing regression of the disease or condition; or

(iv) relieving the symptoms resulting from the disease or condition, i.e., relieving pain without addressing the underlying disease or condition.

The term “effective amount” refers to an amount of a compound of the invention or other active ingredient sufficient to provide a therapeutic or prophylactic benefit in the treatment or prevention of a disease or to delay or minimize symptoms associated with a disease. Further, a therapeutically effective amount with respect to a compound of the invention means that amount of therapeutic agent alone, or in combination with other therapies, that provides a therapeutic benefit in the treatment or prevention of a disease. Used in connection with a compound of the invention, the term can encompass an amount that improves overall therapy, reduces or avoids symptoms or causes of disease, or enhances the therapeutic efficacy or synergies with another therapeutic agent.

A “therapeutically effective amount (or dose)” of a compound refers to that amount sufficient to result in amelioration of one or more symptoms of the disease being treated in a statistically significant manner. When referring to an individual active ingredient administered alone, a therapeutically effective dose refers to that ingredient alone. When referring to a combination, a therapeutically effective dose refers to combined amounts of the active ingredients that result in the therapeutic effect, whether administered serially or simultaneously.

The term “pharmaceutically acceptable” refers to molecular entities and compositions that do not produce allergic or other serious adverse reactions when administered to a subject using routes well-known in the art.

A “patient” or subject” or “subject in need” refers to a subject at risk of developing, suspected to be suffering from, or suffering from, a disease, disorder or condition (e.g., an eIF4A dependent condition) that is amenable to treatment or amelioration with a compound or a composition thereof provided herein. Thus, subjects in need of administration of therapeutic agents as described herein include, but are not limited to, subjects suspected of having an eIF4A dependent condition (e.g., a hyperproliferative disease such as cancer), subjects with an existing eIF4A dependent condition, or subjects receiving a vaccine directed to treating an eIF4A dependent condition. A “subject in need” includes any organism capable of developing an eIF4A dependent condition or being infected, such as primates, (e.g., humans, monkeys and apes), and non-primates such as domestic animals, including laboratory animals and household pets, livestock, show animals, zoo specimens, or other animals, and non-domestic animals, such as wildlife or the like. For example, a subject or a subject in need may be a human, a non-human primate, cow, horse, sheep, lamb, pig, chicken, turkey, quail, dog, cat, rabbit, horse, mouse, rat, guinea pig, or the like. In specific embodiments, a subject or a subject in need is a human, such as a human infant, child, adolescent or adult.

A “biological sample” or “sample” includes blood and blood fractions or products (e.g., serum, plasma, platelets, red blood cells, or the like); sputum or saliva; kidney, lung, liver, heart, brain, nervous tissue, thyroid, eye, skeletal muscle, cartilage, or bone tissue; cultured cells, e.g., primary cultures, explants, and transformed cells, stem cells, stool, urine, etc. Such biological samples (e.g., disease samples or normal samples) also include sections of tissues, such as a biopsy or autopsy sample, frozen sections taken for histologic purposes, or cells or other biological material used to model disease or to be representative of a pathogenic state. In certain embodiments, a biological sample is obtained from a subject, e.g., a eukaryotic organism, most preferably a mammal such as a primate, e.g., chimpanzee or human; cow; dog; cat; rodent, e.g., guinea pig, rat, or mouse; rabbit; bird; reptile; or fish.

In certain embodiments, the present disclosure provides a method for ameliorating or treating an eIF4A dependent condition in a subject in need thereof comprising administering to the subject a therapeutically effective amount of at least one eukaryotic translation initiation factor 4A (eIF4A) inhibitor and a therapeutically effective amount of at least one cyclin-dependent kinase (CDK) inhibitor.

In certain embodiments, the eIF4A dependent condition is a disease of uncontrolled cell growth, proliferation and/or survival. In some aspects, the eIF4A dependent condition is a hyperproliferative disease. In some embodiments, the hyperproliferative disease is cancer. In other embodiments, the hyperproliferative disease comprises an autoimmune disease or an inflammatory disease. In specific aspects, the eIF4A dependent condition is cancer.

In certain embodiments, the cancer includes, but is not limited to, solid tumor, colorectal cancer, bladder cancer, gastric cancer, thyroid cancer, esophageal cancer, head and neck cancer, brain cancer, malignant glioma, fibrotic diseases, glioblastoma, hepatocellular cancers, thyroid cancer, lung cancer, non-small cell lung cancer (NSCLC), small cell lung cancer, melanoma, multiple melanoma, myeloma, pancreatic cancer, pancreatic carcinoma, renal cell carcinoma, renal cancer, cervical cancer, urothelial cancer, prostate cancer, castration-resistant prostate cancer, ovarian cancer, breast cancer, triple-negative breast cancer, leukemia, acute myeloid leukemia, Hodgkins lymphoma, non-Hodgkins lymphoma, mantle cell lymphoma, B-cell lymphoma, T-cell lymphoma, hairy cell lymphoma, diffuse large B-cell lymphoma, Burkitt's lymphoma, multiple myeloma, and liposarcoma. In specific embodiments, the cancer is breast cancer. In certain aspects, the breast cancer is estrogen receptor-positive (ER⁺) breast cancer. In other embodiments, the cancer is non-small cell lung cancer (NSCLC). In particular aspects, the non-small cell lung cancer (NSCLC) is Kirsten rat sarcoma viral oncogene homolog (KRAS)-mutant NSCLC. In yet other embodiments, the cancer is colorectal cancer.

In certain embodiments, the at least one CDK inhibitor inhibits cyclin-dependent kinase (CDK) proteins, such as CDK1, CDK2, CDK3, CDK4, CDK5, CDK 6, CDK 7, CDK 8, CDK 9, CDK 10, CDK11, and/or CDK 12. In specific embodiments, the CDK inhibitor inhibits CDK4, CDK6, or both CDK4 and CDK6. Thus, in certain embodiments, the at least one CDK inhibitor is a CDK4/6 inhibitor. Exemplary CDK4/6 inhibitors of this disclosure include, but are not limited to, palbociclib, ribociclib, abemaciclib, trilaciclib, flavopiridol (alvocidib), G1T28-1, G1T38, ON123300, AT7519HCl, P276-00, AT7519, JNJ-7706621, SHR6390, PF-06873600, and derivatives thereof. In specific embodiments, the CDK4/6 inhibitor is palbociclib, ribociclib, or abemaciclib. In certain embodiments, the CDK4/6 inhibitor is palbociclib. In other embodiments, the CDK4/6 inhibitor is ribociclib. In additional embodiments, the CDK4/6 inhibitor is abemaciclib. These compounds are discussed in greater detail in U.S. Pat. Nos. 6,936,612, 8,324,225, and 7,855,211, which compounds and synthetic methods of making such compounds disclosed therein are incorporated herein by reference in their entirety.

Exemplary site-directed eIF4A inhibitors of this disclosure include compounds according to Formula I:

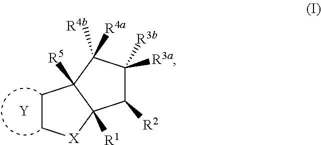

or stereoisomers, tautomers or pharmaceutically acceptable salts thereof,

wherein:

X is CR⁶R⁷, O, S, NH, N(C₁-C₈)alkyl, C(O), C═CR⁶R⁷, N(CO)R⁸, S(O) or S(O)₂;

Y is a 5-membered heteroaryl or a 6-membered aryl or heteroaryl;

R¹ and R² independently are aryl, heterocyclyl, heteroaryl or cycloalkyl;

R^{3a}, R^{3b}, R^{4a} and R^{4b} independently are H, halogen, CN, C₁-C₈(alkyl), (C₁-C₈)haloalkyl, C₂-C₈(alkenyl), (C₂-C₈)alkynyl, OR⁹, NHR⁹, NR⁹R⁹, [(C₁-C₈)alkylene]OR⁹, [(C₁-C₈)alkylene]NHR⁹, [(C₁-C₈)alkylene]NR⁹R⁹, C(O)R⁸, C(O)NHR⁹, C(O)NR⁹R⁹, C(O)[(C₁-C₈)alkylene]NHR⁹, C(O)[(C₁-C₈)alkylene]NR⁹R⁹, CO₂R⁹, C(S)NHR⁹, C(S)NR⁹R⁹, SR⁹, S(O)R⁹, SO₂R⁹, SO₂NHR⁹, SO₂NR⁹R⁹, NH(CO)R⁸, NR⁹(CO)R⁸, NH(CO)NHR⁹, NH(CO)NR⁹R⁹, NR⁹(CO)NHR⁹, NR⁹(CO)NR⁹R⁹, P(O)(OH)(OR⁹), P(O)(OR⁹)(OR⁹), aryl, heteroaryl, cycloalkyl or heterocyclyl;

R^{3a} and R^{3b}, and R^{4a} and R^{4b} independently combine to form oxo or alkenyl, or a cycloalkyl or heterocyclyl ring; or

R^{3a} and R^{4a}, R^{3b} and R^{4b} or R^{4a} and R⁵ together with the carbon atom to which they are attached form a cycloalkyl or heterocyclyl ring; or

R² and R^{3a} together with the carbon atom to which they are attached form a bicyclic ring system;

R⁵ is H, halogen, OH, CN, N3, SR⁹, (C₁-C₈)alkyl, (C₁-C₈)haloalkyl, O(C₁-C₈)alkyl, O(C₁-C₈)haloalkyl, (C₂-C₈)alkynyl, NHC(O)(C₁-C₈)alkyl or heteroaryl;

R⁶ and R⁷ independently are H, CN, halogen, OR⁹, SR⁹, (C₁-C₈)alkyl, NH(R⁹) or NR⁹R⁹;

R⁸ is H, (C₁-C₈)alkyl, (C₁-C₈)haloalkyl, O(C₁-C₈)alkyl, O(C₁-C₈)haloalkyl, cycloalkyl, O(cycloalkyl), heterocyclyl, O(heterocyclyl), aryl, O(aryl), heteroaryl or O(heteroaryl);

R⁹ is H, (C₁-C₈)alkyl, (C₁-C₈)haloalkyl, cycloalkyl, heterocyclyl, [(C₁-C₈)alkylene] heterocyclyl, aryl, [(C₁-C₈)alkylene] aryl or heteroaryl;

wherein the two R⁹'s together with the nitrogen atom to which they are attached of NR⁹R⁹, [(C₁-C₈)alkylene]NR⁹R⁹, C(O)NR⁹R⁹, C(O)[(C₁-C₈)alkylene]NR⁹R⁹, C(S)NR⁹R⁹, SO₂NR⁹R⁹, NH(CO)NR⁹R⁹ or NR⁹(CO)NR⁹R⁹, optionally form a heterocyclyl ring;

wherein any alkyl, alkenyl, cycloalkyl, heterocyclyl, heteroaryl or aryl is optionally substituted with 1, 2, or 3 groups selected from OH, CN, SH, SO₂NH₂, SO₂(C₁-C₄)alkyl, SO₂NH(C₁-C₄)alkyl, halogen, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, NH₂—C(O)-alkylene, NH(Me)-C(O)-alkylene, CH₂—C(O)-lower alkyl, C(O)-lower alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; or

wherein any alkyl, cycloalkyl or heterocyclyl is optionally substituted with oxo;

“m” and “p” are 1, 2, 3, 4, 5 or 6; and

wherein when Y is a 6-membered aryl then X is not O.

In certain embodiments, X is O.

In some embodiments, Y is a 6-membered heteroaryl wherein A¹ is N, A² is CR¹¹, A³ is CR¹² and A⁴ is CR¹³, wherein R¹¹, R¹² and R¹³ independently are H, CN, halogen or OR⁹.

In other embodiments, Y is a 6-membered heteroaryl wherein A² is N, A¹ is CR¹⁰, A³ is CR¹² and A⁴ is CR¹³, wherein R¹⁰, R¹² and R¹³ independently are H, CN, halogen or OR⁹.

In yet other embodiments, Y is a 6-membered heteroaryl wherein A³ is N, A¹ is CR¹⁰, A² is CR¹¹ and A⁴ is CR¹³, wherein R¹⁰, R¹¹ and R¹³ independently are H, CN, halogen or OR⁹.

In still other embodiments, Y is a 6-membered heteroaryl wherein A⁴ is N, A¹ is CR¹⁰, A² is CR¹¹ and A³ is CR¹², wherein R¹⁰, R¹¹ and R¹² independently are H, CN, halogen or OR⁹.

In additional embodiments, Y is a 6-membered heteroaryl wherein A² and A⁴ are N, A¹ is CR¹⁰ and A³ is CR¹², wherein R¹⁰ and R¹² independently are H, CN, halogen or OR⁹.

In other embodiments, Y is a 5-membered heteroaryl wherein B¹ and B³ are N or S and B² is CR¹⁴, wherein R¹⁴ is H, CN, halogen or OR⁹.

In yet other embodiments, Y is a 5-membered heteroaryl wherein B¹ is N, B² is NR¹⁵ and B³ is CR¹⁴, wherein R¹⁴ is H and R¹⁵ is OR⁹ or C₁-C₆(alkyl).

In certain embodiments, R¹ and R² are aryl.

In some embodiments, R^{3a}, R^{3b}, R^{4a} and R^{4b} independently are H, halogen, C₁-C₈(alkyl), (C₁-C₈)haloalkyl, OH, CN, [(C₁-C₈)alkylene]OR⁹, [(C₁-C₈)alkylene]NHR⁹, [(C₁-C₈)alkylene]NR⁹R⁹, C(O)NH₂, C(O)NHR⁹, C(O)NR⁹R⁹, C(O)R⁹, CO₂R⁹, C(S)NH₂, S(O)R⁹, SO₂R⁹, SO₂NHR⁹, SO₂NR⁹R⁹, heteroaryl or cycloalkyl, wherein R⁹ is a C₁-C₈(alkyl) or (C₁-C₈)haloalkyl, or wherein the two R⁹'s together with the nitrogen atom to which they are attached of [(C₁-C₈)alkylene]NR⁹R⁹, C(O)NR⁹R⁹ or SO₂NR⁹R⁹ optionally form a heterocyclyl ring.

In other embodiments, R^{3b} is [(C₁-C₈)alkylene]NHR⁹ or [(C₁-C₈)alkylene]NR⁹R⁹, wherein R⁹ is C₁-C₈(alkyl) or (C₁-C₈)haloalkyl, or wherein the two R⁹'s together with the nitrogen atom to which they are attached of [(C₁-C₈)alkylene]NR⁹R⁹ optionally form a heterocyclyl ring.

In other embodiments, R^{4b} is OH.

In additional embodiments, R^{4a} and R^{4b} combine to form oxo or alkenyl.

In yet other embodiments, R^{3a} and R^{4a}, R^{3b} and R^{4b} or R^{4a} and R⁵ together with the carbon atom to which they are attached form a cycloalkyl or heterocyclyl ring.

In certain embodiments, R⁵ is OH.

In some embodiments, R⁶ and R⁷ are H or C₁-C₈(alkyl).

In some embodiments, R⁹ is H or C₁-C₈(alkyl). In other embodiments, R⁹ is CH₃.

In some embodiments, the 6-membered aryl or heteroaryl is

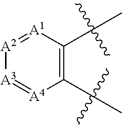

wherein

A¹ is N or CR¹⁰;

A² is N or CR¹¹;

A³ is N or CR¹²;

A⁴ is N or CR¹³; and

R¹⁰, R¹¹, R¹² and R¹³ independently are H, halogen, C₁-C₈(alkyl), (C₁-C₈)haloalkyl, C(O)O(C₁-C₈)alkyl, C(O)(C₁-C₈)alkyl, SO₂(C₁-C₈)alkyl, C₂-C₈(alkenyl), (C₂-C₈)alkynyl, OR⁹, NHR⁹, NR⁹R⁹, CN, [(C₁-C₈)alkylene]OR⁹, [(C₁-C₈)alkylene]NHR⁹, [(C₁-C₈)alkylene]NR⁹R⁹, C(O)R⁸, C(O)NHR⁹, C(O)NR⁹R⁹, C(O)[(C₁-C₈)alkylene]NHR⁹, C(O)[(C₁-C₈)alkylene]NR⁹R⁹, CO₂R⁹, C(S)NHR⁹, C(S)NR⁹R⁹, SR⁹, S(O)R⁹, SO₂R⁹, SO₂NHR⁹, SO₂NR⁹R⁹, NH(CO)R⁸, NR⁹(CO)R⁸, NH(CO)NHR⁹, NH(CO)NR⁹R⁹, NR⁹(CO)NHR⁹, NR⁹(CO)NR⁹R⁹, P(O)(OH)(OR⁹), P(O)(OR⁹)(OR⁹), aryl, heteroaryl, cycloalkyl or heterocyclyl.

In certain embodiments, the 5-membered heteroaryl is

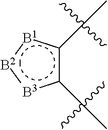

wherein any two of B¹, B² and B³ are CR¹⁴ and N and the remaining B ring atom is N(R¹⁵) or S, wherein R¹⁴ is H, CN, halogen, OR⁹, SR⁹, (C₁-C₈)alkyl, C(O)O(C₁-C₈)alkyl, C(O)(C₁-C₈)alkyl, SO₂(C₁-C₈)alkyl, SO₂NR⁹R⁹, C(O)NR⁹R⁹, NR⁹R⁹ or NR⁹C(O)R⁸, and R¹⁵ is H or (C₁-C₈)alkyl.

In certain aspects, compounds according to Formula I may be isotopically-labeled by having one or more atoms replaced by an atom having a different atomic mass or mass number. Examples of isotopes that can be incorporated into compounds of according to Formula I include isotopes of hydrogen, carbon, nitrogen, oxygen, phosphorous, fluorine, chlorine, or iodine.

Illustrative of such isotopes are ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds can be used to measure the biodistribution, tissue concentration and the kinetics of transport and excretion from biological tissues including a subject to which such a labeled compound is administered. Labeled compounds are also used to determine therapeutic effectiveness, the site or mode of action, and the binding affinity of a candidate therapeutic to a pharmacologically important target. Certain radioactive-labeled compounds according to Formula I, therefore, are useful in drug and/or tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e. ²H, affords certain therapeutic advantages resulting from the greater metabolic stability, for example, increased in vivo half-life of compounds containing deuterium. Substitution of hydrogen with deuterium may reduce dose required for therapeutic effect, and hence may be preferred in a discovery or clinical setting.

Substitution with positron emitting isotopes, such as ¹¹C, ¹⁸F, ¹⁵O and ¹³N, provides labeled analogs of the inventive compounds that are useful in Positron Emission Tomography (PET) studies, e.g., for examining substrate receptor occupancy. Isotopically-labeled compounds according to Formula I, can generally be prepared by conventional techniques known to those skilled in the art or by processes analogous to those described in the Preparations and Examples section as set out below using an appropriate isotopic-labeling reagent.

In certain embodiments, methods disclosed herein also encompass use or activity of in vivo metabolic products of compounds according to Formula I. Such products may result from, for example, the oxidation, reduction, hydrolysis, amidation, esterification, and like processes primarily due to enzymatic activity upon administration of a compound of the invention. Accordingly, the presently disclosed methods include use of compounds that are produced as by-products of enzymatic or non-enzymatic activity on an eIF4A inhibitor following the administration of such a compound to a mammal for a period of time sufficient to yield a metabolic product. Metabolic products, particularly pharmaceutically active metabolites are typically identified by administering a radiolabeled compound of the invention in a detectable dose to a subject, such as rat, mouse, guinea pig, monkey, or human, for a sufficient period of time during which metabolism occurs, and isolating the metabolic products from urine, blood or other biological samples that are obtained from the subject receiving the radiolabeled compound.

Further examples of eIF4A inhibitors include compounds as disclosed in U.S. Pat. No. 9,957,277, which compounds and synthetic methods of making such compounds disclosed therein are incorporated herein by reference in their entirety.

In specific embodiments, the eIF4A inhibitor is a compound according to the following formula:

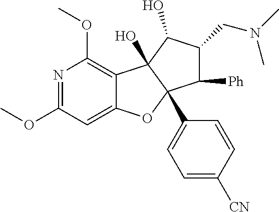

or a stereoisomer, tautomer, or pharmaceutically acceptable salt thereof. The terms “Cpd. No. 231F,” “231F,” and “eFT226” are used interchangeably herein to refer to this compound.

In additional embodiments, the present disclosure provides a method for ameliorating or treating a cancer in a subject in need thereof comprising administering to the subject a therapeutically effective amount of an eIF4A inhibitor and a therapeutically effective amount of a CDK4/6 inhibitor, wherein the eIF4A inhibitor is a compound according to the following formula:

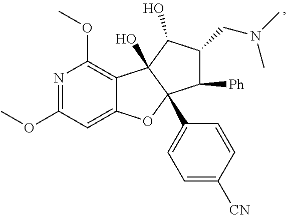

or a stereoisomer, tautomer, or pharmaceutically acceptable salt thereof. The CDK4/6 inhibitor may include, but is not limited to, palbociclib, ribociclib, and abemaciclib.

In some embodiments, the at least one eIF4A inhibitor and/or the at least one CDK inhibitor (e.g., a CDK4/6 inhibitor) is administered to a subject in need thereof at least once every day, every 2 days, every 3 days, every 4 days, every 5 days, every 6 days, every week, every 2 weeks, every 3 weeks, every month, every 2 months, every 3 months, every 4 months, every 5 months, every 6 months, every 7 months, every 8 months, every 9 months, every 10 months, every 11 months, every 1 year, every 2 years, every 3 years, every 4 years, or every 5 years. In additional embodiments, the at least one eIF4A inhibitor and/or the at least one CDK inhibitor (e.g., a CDK4/6 inhibitor) is administered to a subject in need thereof for up to at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 60 days, 90 days, 120 days, 150 days, 180 days, or 365 days.

At least one eukaryotic translation initiation factor 4A (eIF4A) inhibitor and at least one cyclin-dependent kinase (CDK) inhibitor, wherein the at least one eIF4A inhibitor comprises a compound in accordance with Formula I:

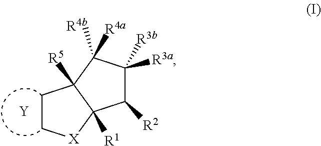

or stereoisomers, tautomers or pharmaceutically acceptable salts thereof,

wherein:

X is CR⁶R⁷, O, S, NH, N(C₁-C₈)alkyl, C(O), C═CR⁶R⁷, N(CO)R⁸, S(O) or S(O)₂;

Y is a 5-membered heteroaryl or a 6-membered aryl or heteroaryl;

R¹ and R² independently are aryl, heterocyclyl, heteroaryl or cycloalkyl;

R^{3a}, R^{3b}, R^{4a} and R^{4b} independently are H, halogen, CN, C₁-C₈(alkyl), (C₁-C₈)haloalkyl, C₂-C₈(alkenyl), (C₂-C₈)alkynyl, OR⁹, NHR⁹, NR⁹R⁹, [(C₁-C₈)alkylene]OR⁹, [(C₁-C₈)alkylene]NHR⁹, [(C₁-C₈)alkylene]NR⁹R⁹, C(O)R⁸, C(O)NHR⁹, C(O)NR⁹R⁹, C(O)[(C₁-C₈)alkylene]NHR⁹, C(O)[(C₁-C₈)alkylene]NR⁹R⁹, CO₂R⁹, C(S)NHR⁹, C(S)NR⁹R⁹, SR⁹, S(O)R⁹, SO₂R⁹, SO₂NHR⁹, SO₂NR⁹R⁹, NH(CO)R⁸, NR⁹(CO)R⁸, NH(CO)NHR⁹, NH(CO)NR⁹R⁹, NR⁹(CO)NHR⁹, NR⁹(CO)NR⁹R⁹, P(O)(OH)(OR⁹), P(O)(OR⁹)(OR⁹), aryl, heteroaryl, cycloalkyl or heterocyclyl;

R^{3a} and R^{3b}, and R^{4a} and R^{4b} independently combine to form oxo or alkenyl, or a cycloalkyl or heterocyclyl ring; or

R^{3a} and R^{4a}, R^{3b} and R^{4b} or R^{4a} and R⁵ together with the carbon atom to which they are attached form a cycloalkyl or heterocyclyl ring; or

R² and R^{3a} together with the carbon atom to which they are attached form a bicyclic ring system;

R⁵ is H, halogen, OH, CN, N3, SR⁹, (C₁-C₈)alkyl, (C₁-C₈)haloalkyl, O(C₁-C₈)alkyl, O(C₁-C₈)haloalkyl, (C₂-C₈)alkynyl, NHC(O)(C₁-C₈)alkyl or heteroaryl;

R⁶ and R⁷ independently are H, CN, halogen, OR⁹, SR⁹, (C₁-C₈)alkyl, NH(R⁹) or NR⁹R⁹;

R⁸ is H, (C₁-C₈)alkyl, (C₁-C₈)haloalkyl, O(C₁-C₈)alkyl, O(C₁-C₈)haloalkyl, cycloalkyl, O(cycloalkyl), heterocyclyl, O(heterocyclyl), aryl, O(aryl), heteroaryl or O(heteroaryl);

R⁹ is H, (C₁-C₈)alkyl, (C₁-C₈)haloalkyl, cycloalkyl, heterocyclyl, [(C₁-C₈)alkylene] heterocyclyl, aryl, [(C₁-C₈)alkylene] aryl or heteroaryl;

wherein the two R⁹'s together with the nitrogen atom to which they are attached of NR⁹R⁹, [(C₁-C₈)alkylene]NR⁹R⁹, C(O)NR⁹R⁹, C(O)[(C₁-C₈)alkylene]NR⁹R⁹, C(S)NR⁹R⁹, SO₂NR⁹R⁹, NH(CO)NR⁹R⁹ or NR⁹(CO)NR⁹R⁹, optionally form a heterocyclyl ring;

wherein any alkyl, alkenyl, cycloalkyl, heterocyclyl, heteroaryl or aryl is optionally substituted with 1, 2, or 3 groups selected from OH, CN, SH, SO₂NH₂, SO₂(C₁-C₄)alkyl, SO₂NH(C₁-C₄)alkyl, halogen, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, NH₂—C(O)-alkylene, NH(Me)-C(O)-alkylene, CH₂—C(O)-lower alkyl, C(O)-lower alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; or

wherein any alkyl, cycloalkyl or heterocyclyl is optionally substituted with oxo;

“m” and “p” are 1, 2, 3, 4, 5 or 6; and

wherein when Y is a 6-membered aryl then X is not O,
for use in the manufacture of a medicament to ameliorate or treat an eIF4A dependent condition in a subject in need thereof. In another embodiment the at least one eukaryotic translation initiation factor 4A (eIF4A) inhibitor is eFT226 and the at least one cyclin-dependent kinase is selected from palbociclib, ribociclib, abemaciclib, trilaciclib, flavopiridol (alvocidib), G1T28-1, G1T38, ON123300, AT7519HCl, P276-00, AT7519, JNJ-7706621, SHR6390, PF-06873600, and derivatives thereof. In another embodiment the at least one eukaryotic translation initiation factor 4A (eIF4A) inhibitor is eFT226 and the cyclin-dependent kinase is selected from palbociclib.

In certain embodiments, the at least one eIF4A inhibitor and/or the at least one CDK inhibitor is administered to a subject in need thereof via a route including, but not limited to, orally, intravenously, intramuscularly, transarterially, intraperitoneally, intranasally, subcutaneously, endoscopically, transdermally, or intrathecally. In specific embodiments, the at least one eIF4A inhibitor is administered to the subject intravenously. In other aspects, the at least one CDK inhibitor is administered to the subject orally.

In certain embodiments, the at least one eIF4A inhibitor is administered to a subject in need thereof in the range from about 0.01 mg/Kg to about 100 mg/Kg. In specific aspects, the at least one eIF4A inhibitor is administered to the subject at about 0.1 mg/Kg. In some aspects, the at least one eIF4A inhibitor is administered to the subject at about 0.1 mg/Kg, every 4 days, for about 25 days. In specific aspects, the at least one eIF4A inhibitor is administered to the subject intravenously at about 0.1 mg/Kg, every 4 days, for about 25 days.

In other embodiments, the at least one CDK inhibitor (e.g., a CDK4/6 inhibitor) is administered to the subject in the range from about 0.01 mg/Kg to about 100 mg/Kg. In specific aspects, the at least one CDK inhibitor (e.g., a CDK4/6 inhibitor) is administered to the subject at about 30 mg/Kg. In additional aspects, the at least one CDK inhibitor is administered to the subject at about 30 mg/Kg, every day, for about 25 days. In specific aspects, the at least one CDK inhibitor is administered to the subject orally at about 30 mg/Kg, every day, for about 25 days.

The at least one eIF4A inhibitor and the at least one CDKinhibitor (e.g., a CDK4/6 inhibitor) may be administered serially, simultaneously, or concurrently to a subject in need thereof. Thus, in some embodiments the eIF4A inhibitor is administered at the same time as the CDK inhibitor (e.g., a CDK4/6 inhibitor). In other embodiments the CDK inhibitor (e.g., a CDK4/6 inhibitor) is administered and after a sufficient period of time the eIF4A inhibitor is administered. In additional embodiments the eIF4A inhibitor is administered and after a sufficient period of time the CDK inhibitor (e.g., a CDK4/6 inhibitor) is administered. When administering serially, the at least one eIF4A inhibitor is formulated in a separate composition from the at least one CDK inhibitor. In certain aspects, when administering simultaneously or concurrently, the at least one eIF4A inhibitor and the at least one CDK inhibitor are formulated in the same composition. In other aspects, when administering simultaneously or concurrently, the at least one eIF4A inhibitor is formulated in a separate composition from the at least one CDK inhibitor. In any of these embodiments, the at least one eIF4A inhibitor and the at least one CDKinhibitor (e.g., a CDK4/6 inhibitor) can be administered as a single dose unit or administered as a single dose unit a plurality of times (daily, weekly, biweekly, monthly, biannually, annually, etc., or any combination thereof).

The methods disclosed herein also provide use of pharmaceutically acceptable salt forms of the eIF4A inhibitors (such as Formula I compounds) and the CDKinhibitors (e.g., a CDK4/6 inhibitor) described herein. Encompassed within the scope of this disclosure are uses of both acid and base addition salts that are formed by contacting a pharmaceutically suitable acid or a pharmaceutically suitable base with an eIF4A inhibitor and/or a CDK inhibitor.

In some embodiments, an eIF4A inhibitor is a specific eIF4A inhibitor of any one of Formulae disclosed herein, which is formulated as a pharmaceutical composition in an amount effective to treat a particular disease or condition of interest (e.g., cancer) upon administration of the pharmaceutical composition to a subject (e.g., human). In particular embodiments, a pharmaceutical composition comprises a therapeutically effective amount of at least one eIF4A inhibitor as described herein, and a pharmaceutically acceptable carrier, diluent or excipient.

In other embodiments, a CDK inhibitor (e.g., a CDK4/6 inhibitor) as described herein is formulated as a pharmaceutical composition in an amount effective to treat a particular disease or condition of interest (e.g., cancer) upon administration of the pharmaceutical composition to a subject (e.g., human). In particular embodiments, a pharmaceutical composition comprises a therapeutically effective amount of at least one CDK inhibitor (e.g., a CDK4/6 inhibitor) as described herein, and a pharmaceutically acceptable carrier, diluent or excipient.

Thus, in certain embodiments, a pharmaceutical composition of the disclosure comprises a therapeutically effective amount of at least one eIF4A inhibitor as described herein and a pharmaceutically acceptable carrier, diluent or excipient. In other embodiments, a pharmaceutical composition of the disclosure comprises a therapeutically effective amount of at least one CDK inhibitor (e.g., a CDK4/6 inhibitor) as described herein and a pharmaceutically acceptable carrier, diluent or excipient. In additional embodiments, a pharmaceutical composition of the disclosure comprises a therapeutically effective amount of at least one eIF4A inhibitor and a therapeutically effective amount of at least one CDK inhibitor (e.g., a CDK4/6 inhibitor) as described herein, and a pharmaceutically acceptable carrier, diluent or excipient.

In this regard, a “pharmaceutically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier that has been approved by the United States Food and Drug Administration as being acceptable for use in humans or domestic animals. A pharmaceutically acceptable carrier includes any solvent, dispersion media, or coating that are physiologically compatible and that preferably do not interfere with or otherwise inhibit the activity of the therapeutic agent. Thus, pharmaceutically acceptable carriers can contain one or more physiologically acceptable compound(s) that act, for example, to stabilize the composition or to increase or decrease the absorption of the active agent(s). Preferably, a carrier is suitable for intravenous, intramuscular, oral, intraperitoneal, transdermal, topical, or subcutaneous administration. Physiologically acceptable carriers can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are well-known and generally described in, for example, Remington: The Science and Practice of Pharmacy, 21st Edition, Philadelphia, Pa. Lippincott Williams & Wilkins, 2005. Various pharmaceutically acceptable excipients are well-known in the art and can be found in, for example, Handbook of Pharmaceutical Excipients (5^{th} ed., Ed. Rowe et al., Pharmaceutical Press, Washington, D.C.).

A pharmaceutical composition of this disclosure can be prepared by combining or formulating at least one eIF4A inhibitor and/or at least one CDK inhibitor (e.g., a CDK4/6 inhibitor) as described herein with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Exemplary routes of administering such pharmaceutical compositions include oral, topical, transdermal, inhalation, parenteral, sublingual, buccal, rectal, vaginal, and intranasal. The term parenteral, as used herein, includes subcutaneous injections, intravenous, intramuscular, intrasternal injection or infusion techniques. Pharmaceutical compositions of this disclosure are formulated to allow the active ingredients contained therein to be bioavailable upon administration to a patient. Compositions that will be administered to a subject take the form of one or more dosage units, where, for example, a tablet may be a single dosage unit, and a container of at least one eIF4A inhibitor as described herein and/or at least one CDK inhibitor (e.g., a CDK4/6 inhibitor) in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). A composition to be administered will, in any event, contain a therapeutically effective amount of at least one eIF4A inhibitor and/or at least one CDK inhibitor (e.g., a CDK4/6 inhibitor) of this disclosure, or a pharmaceutically acceptable salt thereof, for treatment of a disease or condition of interest in accordance with the teachings herein.

A pharmaceutical composition of an eIF4A inhibitor and/or a CDK inhibitor (e.g., a CDK4/6 inhibitor) of this disclosure may be in the form of a solid or liquid. In one aspect, the carrier(s) are particulate so that the compositions are, for example, in tablet or powder form. The carrier(s) may be liquid, with a composition being, for example, an oral syrup, injectable liquid or an aerosol, which is useful in, for example, inhalatory administration. When intended for oral administration, a pharmaceutical composition of this disclosure is preferably in either solid or liquid form, where semi-solid, semi-liquid, suspension and gel forms are included within the forms considered herein as either solid or liquid.

As a solid composition for oral administration, a pharmaceutical composition of an eIF4A inhibitor and/or a CDK inhibitor (e.g., a CDK4/6 inhibitor) of this disclosure may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

A pharmaceutical composition of an eIF4A inhibitor and/or a CDK inhibitor (e.g., a CDK4/6 inhibitor) of this disclosure may be in the form of a liquid, for example, an elixir, syrup, solution, emulsion or suspension. The liquid may be for oral administration or for delivery by injection, as two examples. When intended for oral administration, compositions contain, in addition to an eIF4A inhibitor and/or a CDK inhibitor (e.g., a CDK4/6 inhibitor) of this disclosure, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of eIF4A inhibitors and/or CDK inhibitors (e.g., CDK4/6 inhibitors) of this disclosure, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. In certain aspects, physiological saline is an adjuvant. An injectable pharmaceutical composition is preferably sterile.

A liquid pharmaceutical composition of an eIF4A inhibitor and/or a CDK inhibitor (e.g., a CDK4/6 inhibitor) of this disclosure intended for either parenteral or oral administration should contain an amount of an eIF4A inhibitor and/or a CDK inhibitor (e.g., a CDK4/6 inhibitor) of this disclosure such that a suitable dosage will be obtained.

A pharmaceutical composition of an eIF4A inhibitor and/or a CDK inhibitor (e.g., a CDK4/6 inhibitor) of this disclosure may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, a composition of an eIF4A inhibitor and/or a CDK inhibitor (e.g., a CDK4/6 inhibitor) of this disclosure may be included with a transdermal patch or iontophoresis device.

The pharmaceutical composition of an eIF4A inhibitor and/or a CDK inhibitor (e.g., a CDK4/6 inhibitor) of this disclosure may be intended for rectal administration, in the form, for example, of a suppository, which will melt in the rectum and release the drug. A composition for rectal administration may contain an oleaginous base as a suitable nonirritating excipient. Such bases include, for example, lanolin, cocoa butter or polyethylene glycol.

The pharmaceutical composition of an eIF4A inhibitor and/or a CDK inhibitor (e.g., a CDK4/6 inhibitor) of this disclosure may include various materials that modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical compositions of this disclosure in solid or liquid form may include an agent that binds to an eIF4A inhibitor and/or a CDK inhibitor (e.g., a CDK4/6 inhibitor) of this disclosure, and thereby assist in the delivery of the compounds. Suitable agents that may act in this capacity include a monoclonal or polyclonal antibody, a protein or a liposome.

A pharmaceutical composition of an eIF4A inhibitor and/or a CDK inhibitor (e.g., a CDK4/6 inhibitor) of this disclosure may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of eIF4A inhibitors and/or CDK inhibitors (e.g., CDK4/6 inhibitors) of this disclosure may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation, may determine preferred aerosol formulations and delivery modes.

A pharmaceutical composition of this disclosure may be prepared by methodology well-known in the pharmaceutical art. For example, a pharmaceutical composition intended to be administered by injection can be prepared by combining an eIF4A inhibitor and/or a CDK inhibitor (e.g., a CDK4/6 inhibitor) of this disclosure with a sterile solvent so as to form a solution. A surfactant may be added to facilitate the formation of a homogeneous solution or suspension. Surfactants are compounds that non-covalently interact with a compound of this disclosure so as to facilitate dissolution or homogeneous suspension of the compound in an aqueous delivery system.

Combination Therapies with Additional Agents

In additional embodiments, the methods of the present disclosure involve combination therapy using at least one eIF4A inhibitor and at least one CDK inhibitor (e.g., a CDK4/6 inhibitor) and at least one additional therapeutic agent.

In further embodiments, the combination of at least one eIF4A inhibitor and at least one CDK inhibitor (e.g., a CDK4/6 inhibitor) described herein can be used in combination with an adjunctive therapy, such as an anti-cancer agent. Anti-cancer agents include chemotherapeutic drugs. A chemotherapeutic agent includes, for example, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), or a DNA repair inhibitor. In further embodiments, the combination of at least one eIF4A inhibitor and at least one CDK inhibitor (e.g., a CDK4/6 inhibitor) described herein is used in combination with a chemotherapeutic agent and a PD-1 specific antibody or binding fragment thereof. In still further embodiments, the combination of at least one eIF4A inhibitor and at least one CDK inhibitor (e.g., a CDK4/6 inhibitor) described herein is used in combination with a chemotherapeutic agent and a PD-L1 specific antibody or binding fragment thereof. In yet further embodiments, the combination of at least one eIF4A inhibitor and at least one CDK inhibitor (e.g., a CDK4/6 inhibitor) described herein is used in combination with a chemotherapeutic agent and a CTLA4 specific antibody or binding fragment thereof, or fusion protein. In yet further embodiments, the combination of at least one eIF4A inhibitor and at least one CDK inhibitor (e.g., a CDK4/6 inhibitor) described herein is used in combination with a chemotherapeutic agent and a LAG3 specific antibody or binding fragment thereof, or fusion protein.

Chemotherapeutic agents include, for example, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (methotrexate, pemetrexed, mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones, eribulin and navelbine; epidipodophyllotoxins (etoposide, teniposide); DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); DNA methyltransferase inhibitors (azacytidine); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkylsulfonates (busulfan), nitrosoureas (carmustine (BCNU) and analogs, streptozocin), triazenes (dacarbazine (DTIC)); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein, pomalidomide) and growth factor inhibitors (vascular endothelial growth factor (VEGF) inhibitors, such as ziv-aflibercept; fibroblast growth factor (FGF) inhibitors); inhibitors of apoptosis protein (IAP) antagonists (birinapant); histone deacetylase (HDAC) inhibitors (vorinostat, romidepsin, chidamide, panobinostat, mocetinostat, abexinostat, belinostat, entinostat, resminostat, givinostat, quisinostat, SB939); proteasome inhibitors (ixazomib); angiotensin receptor blocker; nitric oxide donors; anti-sense oligonucleotides; antibodies (trastuzumab, panitumumab, pertuzumab, cetuximab, adalimumab, golimumab, infliximab, rituximab, ocrelizumab, ofatumumab, obinutuzumab, alemtuzumab, abciximab, atlizumab, daclizumab, denosumab, efalizumab, elotuzumab, rovelizumab, ruplizumab, ustekinumab, visilizumab, gemtuzumab ozogamicin, brentuximb vedotin); chimeric antigen receptors; cell cycle inhibitors (flavopiridol, roscovitine, bryostatin-1) and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); PARP inhibitors (niraparib, olaparib); focal adhesion kinase (FAK) inhibitors (defactinib (VS-6063), VS-4718, VS-6062, GSK2256098); growth factor signal transduction kinase inhibitors (cediranib, galunisertib, rociletinib, vandetanib, afatinib, EGF816, AZD4547); c-Met inhibitors (capmatinib, INC280); ALK inhibitors (ceritinib, crizotinib); mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

In certain embodiments, a chemotherapeutic agent is a B-Raf inhibitor, a MEK inhibitor, a VEGF inhibitor, a VEGFR inhibitor, a tyrosine kinase inhibitor, an anti-mitotic agent, or any combination thereof. In specific embodiments, the chemotherapeutic is vemurafenib, dabrafenib, trametinib, cobimetinib, sunitinib, erlotinib, paclitaxel, docetaxel, or any combination thereof.

In certain embodiments, a therapy that induces or enhances an anti-cancer response, for example, a vaccine, an inhibitor of an immunosuppression signal, a B-Raf inhibitor, a MEK inhibitor, a VEGF inhibitor, a VEGFR inhibitor, a tyrosine kinase inhibitor, a cytotoxic agent, a chemotherapeutic, or any combination thereof, is used in combination with the combination of at least one eIF4A inhibitor and at least one CDK inhibitor (e.g., a CDK4/6 inhibitor) in the treatment and/or amelioration methods described herein, wherein the therapy that induces or enhances an anti-cancer response does not antagonize, reduce, diminish, or decrease the inhibitory activity of the combination of the eIF4A inhibitor and the CDK inhibitor.

The additional therapy or modulator can be administered serially, simultaneously, or concurrently with the combination of at least one eIF4A inhibitor and at least one CDK inhibitor (e.g., a CDK4/6 inhibitor) described herein. When administering serially, the combination of the at least one eIF4A inhibitor and the at least one CDK inhibitor or pharmaceutical compositions thereof are formulated in a separate composition from a second (or third, etc.) therapy, modulator or pharmaceutical compositions thereof. When administering simultaneously or concurrently, a first and second (or third, etc.) therapy or modulator may be formulated in separate compositions or formulated in a single composition. In any of these embodiments, the single or combination therapies can be administered as a single dose unit or administered as a single dose unit a plurality of times (daily, weekly, biweekly, monthly, biannually, annually, etc., or any combination thereof).

In certain embodiments, a combination therapy described herein is used in a method for treating an eIF4A dependent condition. In certain aspects, the eIF4A dependent condition is a disease of uncontrolled cell growth, proliferation and/or survival. In some aspects, the eIF4A dependent condition is a hyperproliferative disease. In specific embodiments, the hyperproliferative disease is cancer. In other embodiments, the hyperproliferative disease comprises an autoimmune disease or an inflammatory disease.

A wide variety of hyperproliferative disorders, including solid tumors and leukemias, are amenable to treatment with the combination of the at least one eIF4A inhibitor and at least one CDK inhibitor (e.g., a CDK4/6 inhibitor) described herein. Exemplary cancers that may be treated by the methods of this disclosure include adenocarcinoma of the breast, prostate, and colon; all forms of bronchogenic carcinoma of the lung; myeloid; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell). Additional representative cancers that may be treated include histiocytic disorders; histiocytosis malignant; immunoproliferative small intestinal disease; plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; chordoma; craniopharyngioma; dysgerminoma; hamartoma; mesenchymoma; mesonephroma; myosarcoma; ameloblastoma; cementoma; odontoma; teratoma; thymoma; and trophoblastic tumor.

Exemplary hematological malignancies include acute lymphoblastic leukemia (ALL), acute myeloid leukemia (AML), chronic myelogenous leukemia (CML), chronic eosinophilic leukemia (CEL), myelodysplastic syndrome (MDS), Hodgkin's lymphoma, non-Hodgkin's lymphoma (NHL) (e.g., follicular lymphoma, diffuse large B-cell lymphoma, or chronic lymphocytic leukemia), or multiple myeloma (MM).

Still further exemplary hyperproliferative disorders include adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; sertoli cell tumor; thecoma; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin; angiokeratoma; angiolymphoid hyperplasia with eosinophilia; angioma sclerosing; angiomatosis; glomangioma; hemangioendothelioma; hemangioma; hemangiopericytoma; hemangio sarcoma; lymphangioma; lymphangiomyoma; lymphangiosarcoma; pinealoma; carcinosarcoma; chondrosarcoma; cystosarcoma phyllodes; fibrosarcoma; hemangio sarcoma; leiomyosarcoma; leukosarcoma; liposarcoma; lymphangiosarcoma; myosarcoma; myxo sarcoma; ovarian carcinoma; rhabdomyosarcoma; sarcoma; neoplasms; nerofibromatosis; and cervical dysplasia.

In certain embodiments, the hyperproliferative disorder is a solid tumor. Non-limiting examples of solid tumors include pancreatic cancer; bladder cancer; colorectal cancer; biliary tract cancer, breast cancer, including metastatic breast cancer; prostate cancer, including androgen-dependent and androgen-independent prostate cancer; renal cancer, including, e.g., metastatic renal cell carcinoma; hepatocellular cancer; lung cancer, including, e.g., non-small cell lung cancer (NSCLC), bronchioloalveolar carcinoma (BAC), and adenocarcinoma of the lung; ovarian cancer, including, e.g., progressive epithelial or primary peritoneal cancer; cervical cancer; gastric cancer; esophageal cancer; head and neck cancer, thymus carcinoma, including, e.g., squamous cell carcinoma of the head and neck; skin cancer, including e.g., malignant melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; neuroblastoma, bone cancer; soft tissue sarcoma; and thyroid carcinoma.

In certain embodiments, the hyperproliferative disease is a solid tumor selected from the group consisting of non-small cell lung cancer (NSCLC), pancreatic cancer, esophageal cancer, squamous cell carcinoma, gastric carcinoma, hepatic carcinoma, colon cancer, and melanoma. In specific embodiments, the solid tumor disease is a non-small cell lung cancer (NSCLC). In certain aspects, the NSCLC is squamous cell carcinoma or adenocarcinoma.

In specific aspects, the hyperproliferative disease is a cancer that includes, but is not limited to, solid tumor, colorectal cancer, bladder cancer, gastric cancer, thyroid cancer, esophageal cancer, head and neck cancer, brain cancer, malignant glioma, fibrotic diseases, glioblastoma, hepatocellular cancers, thyroid cancer, lung cancer, non-small cell lung cancer (NSCLC), small cell lung cancer, melanoma, multiple melanoma, myeloma, pancreatic cancer, pancreatic carcinoma, renal cell carcinoma, renal cancer, cervical cancer, urothelial cancer, prostate cancer, castration-resistant prostate cancer, ovarian cancer, breast cancer, triple-negative breast cancer, leukemia, acute myeloid leukemia, Hodgkins lymphoma, non-Hodgkins lymphoma, mantle cell lymphoma, B-cell lymphoma, T-cell lymphoma, hairy cell lymphoma, diffuse large B-cell lymphoma, Burkitt's lymphoma, multiple myeloma, and liposarcoma. In specific embodiments, the cancer is breast cancer. In certain aspects, the breast cancer is estrogen receptor-positive (ER⁺) breast cancer. In other embodiments, the cancer is non-small cell lung cancer (NSCLC). In particular aspects, the non-small cell lung cancer (NSCLC) is Kirsten rat sarcoma viral oncogene homolog (KRAS)-mutant NSCLC. In yet other embodiments, the cancer is colorectal cancer.

Generally, the therapeutic agents of the disclosure (e.g., the at least one eIF4A inhibitor and the at least one CDK inhibitor) are administered to a subject in need thereof at a therapeutically effective amount or dose. Such a dose may be determined or adjusted depending on various factors including the specific therapeutic agents or pharmaceutical compositions, the routes of administration, the subject's condition, that is, stage of the disease, severity of symptoms caused by the disease, general health status, as well as age, gender, and weight, and other factors apparent to a person skilled in the medical art. Similarly, the dose of the therapeutic for treating an eIF4A dependent condition (e.g., a hyperproliferative disease) may be determined according to parameters understood by a person skilled in the medical art. When referring to a combination, a therapeutically effective dose refers to combined amounts of the active ingredients that result in the therapeutic effect, whether administered serially or simultaneously (in the same formulation or concurrently in separate formulations). Optimal doses may generally be determined using experimental models and/or clinical trials. Design and execution of pre-clinical and clinical studies for a therapeutic agent (including when administered for prophylactic benefit) described herein are well within the skill of a person skilled in the relevant art.

The route of administration of a therapeutic agent of the disclosure can be oral, intraperitoneal, transdermal, subcutaneous, by intravenous or intramuscular injection, by inhalation, topical, intralesional, infusion; liposome-mediated delivery; topical, intrathecal, gingival pocket, rectal, intrabronchial, nasal, transmucosal, intestinal, ocular or otic delivery, or any other methods known in the art. In certain embodiments, the at least one eIF4A inhibitor and/or the at least one CDK inhibitor described herein is administered to a subject in need thereof via a route including, but not limited to, orally, intravenously, intramuscularly, transarterially, intraperitoneally, intranasally, subcutaneously, endoscopically, transdermally, or intrathecally. In specific embodiments, the at least one eIF4A inhibitor is administered to the subject intravenously. In other aspects, the at least one CDK inhibitor is administered to the subject orally.

### EXAMPLES

### Example 1

### eFT226 Blocks Key Cell Cycle Targets

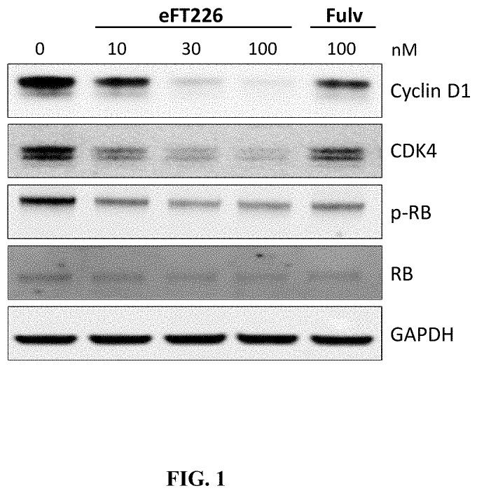

eFT226 is a potent and selective translational regulator that targets eIF4A. eFT226 down-regulates the translation of a unique gene set and displays robust anti-tumor activity across multiple models in vitro and in vivo. The effect of eFT226 on key cell cycle regulators in MDA-MB-361 ER⁺ breast cancer cells was tested. MDA-MB-361 ER⁺ breast cancer cells were treated with varying concentrations of eFT226 (10 nM, 30 nM, and 100 nM) for 24 hours, and analyzed for the expression of the relevant key cell cycle regulators, cyclin D1, CDK4, and phosphorylated retinoblastoma (Rb) protein. As shown in FIG. 1, suppression of cyclin D1 and CDK4 expression was observed in the cell line at all concentrations of eFT226 tested. A concomitant decrease in phosphorylated Rb protein was also observed in the presence of eFT226 (FIG. 1). Thus, FIG. 1 demonstrates that treatment of MDA-MB-361 cells with increasing concentrations of eFT226 for 24 hours results in a dose dependent decrease in protein expression of Cyclin D1, CDK4 and phospho-Rb. Additionally, eFT226 is more effective than Fulvestrant (Fulv) in decreasing Cyclin D1 protein levels. These data indicate that eIF4A inhibitors are effective in targeting these key cell cycle regulators in ER⁺ breast cancer cells.

### Example 2

### Reduced Cell Viability Upon Combination Treatment with eFT226 and Palbociclib

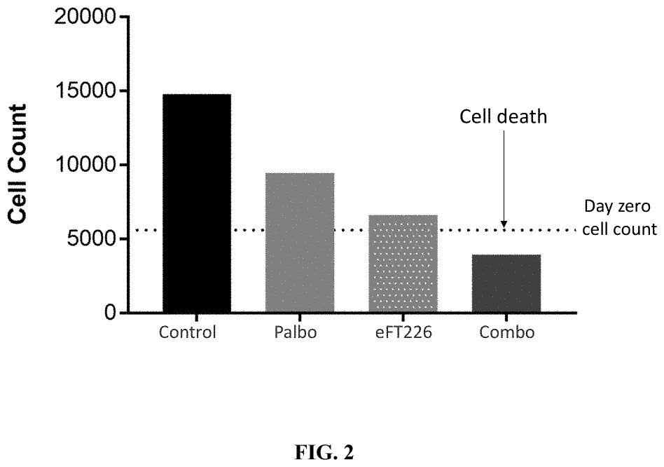

MDA-MB-361 ER⁺ breast cancer cells were seeded at 10,000 cells/well in 24-well plates and treated with DMSO (“control”), Palbociclib (40 nM)(“Palbo”), eFT226 (45 nM) (“eFT226”), or the combination of the two drugs (“Combo”). After 24 hours of treatment, cells were rinsed and treatment with Palbociclib only was continued for 6 days, at which time cell viability was determined. Cells were counted on day 0 and day 6, when the experiment was ended. As shown in FIG. 2, cell viability was reduced in the presence of both palbociclib alone and eFT226 alone in comparison to untreated (control) cells. The combination of palbociclib and eFT226 further repressed cell viability of the MDA-MB-361 cells.

### Example 3

### Combination Treatment Targeting EIF4A and CDK4/6 Synergistically Suppresses ER⁺ Breast Cancer Growth In Vivo

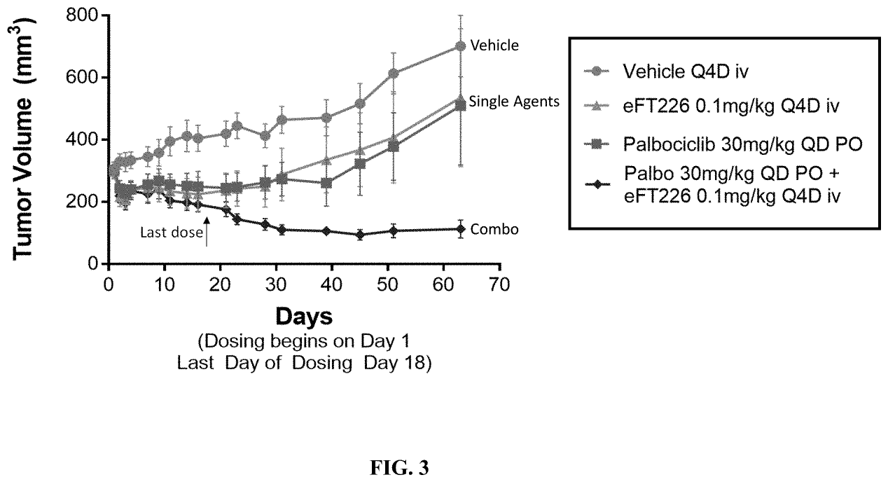

The in vivo effect of a combination treatment using eFT226 and palbociclib on the tumor growth of MDA-MB-361 ER⁺ breast cancer cells was tested. Xenograft experiments were performed by implanting MDA-MB-361 ER⁺ breast cancer cells into athymic mice. Athymic mice implanted with MDA-MB-361 tumor cells were randomized and size-matched into vehicle and treatment groups when the mean tumor size reached ˜300 mm³. The mice were then treated with (1) control vehicle; (2) 0.1 mg/kg of eFT226 administered intravenously every 4 days (Q4D) for a period of 18 days; (3) 30 mg/kg of palbociclib administered orally every day (QD) for a period of 18 days; or (4) 0.1 mg/kg of eFT226 administered intravenously Q4D and 30 mg/kg of palbociclib administered orally QD for a period of 18 days. The effect on the above treatments on tumor volume was monitored. Tumor volume was measured periodically for up to 45 days after the last dose of treatment was administered. As shown in FIG. 3, the control vehicle had no effect on tumor growth. Administration of either eFT226 or palbociclib alone led to a inhibition of tumor growth for about 22 days after the treatment was stopped. However, administration of eFT226 in combination with palbociclib resulted in significant and durable inhibition of tumor growth that persisted 45 days after the last dose (last measurement collected) which was far longer than for either compound alone. These data show that, unexpectedly, eIF4A inhibitors act synergistically with CDK4/6 inhibitors to target ER⁺ breast cancer cells in vivo. It has previously been shown that CDK4/6 inhibitors do not inhibit the active p27-CDK4-cyclin D1 trimers, but instead target monomeric CDK4. It is unknown what factors determine the equilibrium between the CDK4/6 inhibitor-sensitive monomeric CDK4 and the drug resistant p27-CDK4-cyclin D1 trimer. Not to be bound by any one theory, it may be possible that treatment with an eIF4A inhibitor shifts the equilibrium in favor of the monomeric CDK4, thereby allowing the CDK4/6 inhibitors to become more effective.

### Example 4

### Reduced Cell Viability Upon Combination Treatment with eFT226 and Palbociclib in KRAS Mutant Tumors

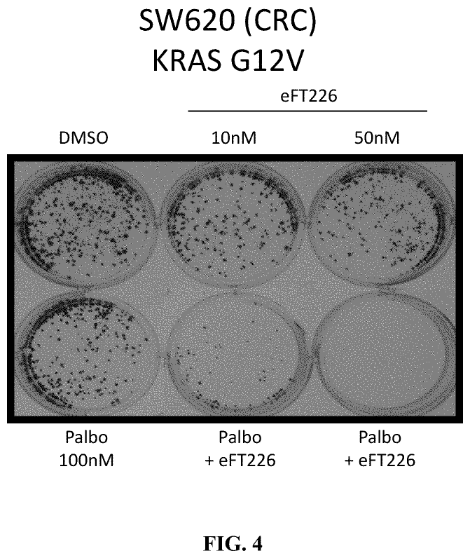

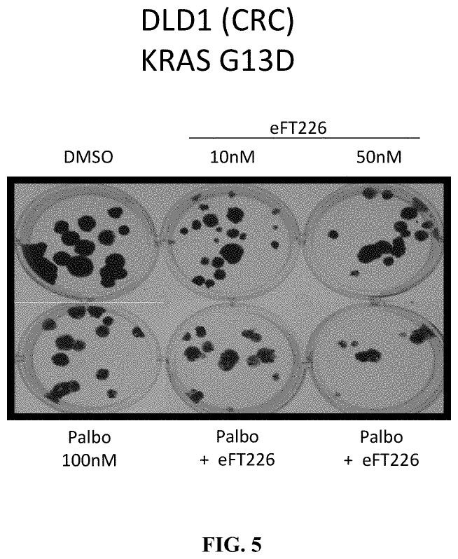

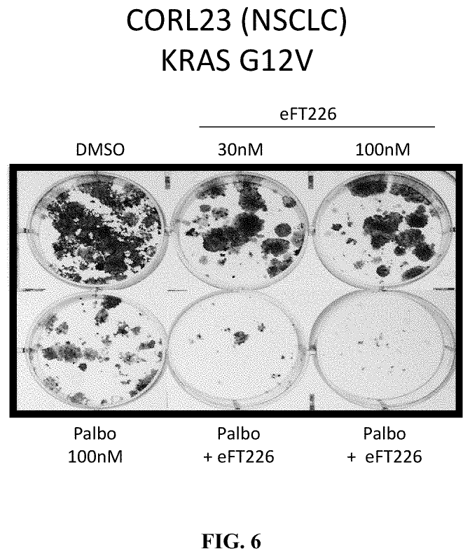

SW620 (KRAS G12V) colorectal cancer cells (FIG. 1), DLD1 (KRAS G13D) colorectal cancer cells (FIG. 2), and CORL23 (KRAS G12V) NSCLC cells (FIG. 3) were seeded at 500-1,000 cells/well in 6-well plates and treated with DMSO control (“DMSO”), Palbociclib (100 nM) (“Palbo”), eFT226 (10 or 50 nM), or the combination of the two drugs (“Palbo+eFT226). After 24 hours of treatment, cells were rinsed and treatment with Palbociclib alone was continued for an additional 13 days. At the end of treatment, cells were rinsed with phosphate buffered saline (PBS) and stained with crystal violet. As shown in FIG. 4, FIG. 5, and FIG. 6 cell viability was reduced in the presence of either Palbociclib or eFT226 alone in comparison to untreated (control) cells. However, the combination of Palbociclib and eFT226 significantly further repressed cell viability for all three KRAS mutant cell lines.

What is claimed is:

1. A method for ameliorating or treating an eIF4A dependent condition in a subject in need thereof comprising administering to the subject a therapeutically effective amount of at least one eukaryotic translation initiation factor 4A (eIF4A) inhibitor and a therapeutically effective amount of at least one cyclin-dependent kinase (CDK) inhibitor, wherein the at least one eIF4A inhibitor comprises a compound in accordance with Formula I: 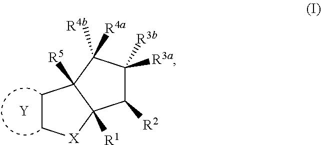
or stereoisomers, tautomers or pharmaceutically acceptable salts thereof,
wherein:
X is CR⁶R⁷, O, S, NH, N(C₁-C₈)alkyl, C(O), C═CR⁶R⁷, N(CO)R⁸, S(O) or S(O)₂;
Y is a 5-membered heteroaryl or a 6-membered aryl or heteroaryl;
R¹ and R² independently are aryl, heterocyclyl, heteroaryl or cycloalkyl;
R^{3a}, R^{3b}, R^{4a} and R^{4b} independently are H, halogen, CN, C₁-C₈(alkyl), (C₁-C₈)haloalkyl, C₂-C₈(alkenyl), (C₂-C₈)alkynyl, OR⁹, NHR⁹, NR⁹R⁹, [(C₁-C₈)alkylene]OR⁹, [(C₁-C₈)alkylene]NHR⁹, [(C₁-C₈)alkylene]NR⁹R⁹, C(O)R⁸, C(O)NHR⁹, C(O)NR⁹R⁹, C(O)[(C₁-C₈)alkylene]NHR⁹, C(O)[(C₁-C₈)alkylene]NR⁹R⁹, CO₂R⁹, C(S)NHR⁹, C(S)NR⁹R⁹, SR⁹, S(O)R⁹, SO₂R⁹, SO₂NHR⁹, SO₂NR⁹R⁹, NH(CO)R⁸, NR⁹(CO)R⁸, NH(CO)NHR⁹, NH(CO)NR⁹R⁹, NR⁹(CO)NHR⁹, NR⁹(CO)NR⁹R⁹, P(O)(OH)(OR⁹), P(O)(OR⁹)(OR⁹), aryl, heteroaryl, cycloalkyl or heterocyclyl;
R^{3a} and R^{3b}, and R^{4a} and R^{4b} independently combine to form oxo or alkenyl, or a cycloalkyl or heterocyclyl ring; or
R^{3a} and R^{4a}, R^{3b} and R^{4b} or R^{4a} and R⁵ together with the carbon atom to which they are attached form a cycloalkyl or heterocyclyl ring; or
R² and R^{3a} together with the carbon atom to which they are attached form a bicyclic ring system;
R⁵ is H, halogen, OH, CN, N₃, SR⁹, (C₁-C₈)alkyl, (C₁-C₈)haloalkyl, O(C₁-C₈)alkyl, O(C₁-C₈)haloalkyl, (C₂-C₈)alkynyl, NHC(O)(C₁-C₈)alkyl or heteroaryl;
R⁶ and R⁷ independently are H, CN, halogen, OR⁹, SR⁹, (C₁-C₈)alkyl, NH(R⁹) or NR⁹R⁹;
R⁸ is H, (C₁-C₈)alkyl, (C₁-C₈)haloalkyl, O(C₁-C₈)alkyl, O(C₁-C₈)haloalkyl, cycloalkyl, O(cycloalkyl), heterocyclyl, O(heterocyclyl), aryl, O(aryl), heteroaryl or O(heteroaryl);
R⁹ is H, (C₁-C₈)alkyl, (C₁-C₈)haloalkyl, cycloalkyl, heterocyclyl, [(C₁-C₈)alkylene] heterocyclyl, aryl, [(C₁-C₈)alkylene] aryl or heteroaryl;
wherein the two R⁹'s together with the nitrogen atom to which they are attached of NR⁹R⁹, [(C₁-C₈)alkylene]NR⁹R⁹, C(O)NR⁹R⁹, C(O)[(C₁-C₈)alkylene]NR⁹R⁹, C(S)NR⁹R⁹, SO₂NR⁹R⁹, NH(CO)NR⁹R⁹ or NR⁹(CO)NR⁹R⁹, optionally form a heterocyclyl ring;
wherein any alkyl, alkenyl, cycloalkyl, heterocyclyl, heteroaryl or aryl is optionally substituted with 1, 2, or 3 groups selected from OH, CN, SH, SO₂NH₂, SO₂(C₁-C₄)alkyl, SO₂NH(C₁-C₄)alkyl, halogen, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, NH₂—C(O)-alkylene, NH(Me)-C(O)-alkylene, CH₂—C(O)-lower alkyl, C(O)-lower alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; or
wherein any alkyl, cycloalkyl or heterocyclyl is optionally substituted with oxo;
“m” and “p” are 1, 2, 3, 4, 5 or 6; and
wherein when Y is a 6-membered aryl then X is not O.

2. The method according to claim 1, wherein the at least one CDK inhibitor is a CDK4/6 inhibitor.

3. The method according to claim 2, wherein the CDK4/6 inhibitor is selected from the group consisting of palbociclib, ribociclib, abemaciclib, trilaciclib, flavopiridol (alvocidib), G1T28-1, G1T38, ON123300, AT7519HCl, P276-00, AT7519, JNJ-7706621, SHR6390, PF-06873600, and derivatives thereof.

4. The method according to claim 3, wherein the CDK4/6 inhibitor is palbociclib, ribociclib, or abemaciclib.

5. The method according to claim 1, wherein the at least one eIF4A inhibitor is a compound according to the following formula: 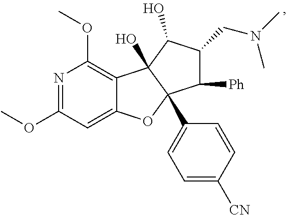
or a stereoisomer, tautomer, or pharmaceutically acceptable salt thereof.

6. The method according to claim 1, wherein the at least one eIF4A inhibitor and/or the at least one CDK inhibitor is administered to the subject via a route selected from the group consisting of orally, intravenously, intramuscularly, transarterially, intraperitoneally, intranasally, subcutaneously, endoscopically, transdermally, or intrathecally.

7. The method according to claim 1, wherein the at least one eIF4A inhibitor is administered to the subject in the range from about 0.01 mg/Kg to about 100 mg/Kg.

8. The method according to claim 7, wherein the at least one eIF4A inhibitor is administered to the subject intravenously at about 0.1 mg/Kg, every 4 days, for about 25 days.

9. The method according to claim 1, wherein the at least one CDK inhibitor is administered to the subject in the range from about 0.01 mg/Kg to about 100 mg/Kg.

10. The method according to claim 9, wherein the at least one CDK inhibitor is administered to the subject orally at about 30 mg/Kg, every day, for about 25 days.

12. The method according to claim 1, wherein the eIF4A dependent condition is a disease of uncontrolled cell growth, proliferation and/or survival.

13. The method according to claim 12, wherein the eIF4A dependent condition is cancer.

14. The method of claim 13, wherein the cancer is selected from the group consisting of solid tumor, colorectal cancer, bladder cancer, gastric cancer, thyroid cancer, esophageal cancer, head and neck cancer, brain cancer, malignant glioma, fibrotic diseases, glioblastoma, hepatocellular cancers, thyroid cancer, lung cancer, non-small cell lung cancer (NSCLC), small cell lung cancer, melanoma, multiple melanoma, myeloma, pancreatic cancer, pancreatic carcinoma, renal cell carcinoma, renal cancer, cervical cancer, urothelial cancer, prostate cancer, castration-resistant prostate cancer, ovarian cancer, breast cancer, triple-negative breast cancer, leukemia, acute myeloid leukemia, Hodgkins lymphoma, non-Hodgkins lymphoma, mantle cell lymphoma, B-cell lymphoma, T-cell lymphoma, hairy cell lymphoma, diffuse large B-cell lymphoma, Burkitt's lymphoma, multiple myeloma, and liposarcoma.

15. The method of claim 14, wherein the cancer is breast cancer.

16. The method of claim 15, wherein the breast cancer is estrogen receptor-positive (ER⁺) breast cancer.

17. The method of claim 14, wherein the cancer is non-small cell lung cancer (NSCLC).

18. The method of claim 17, wherein the non-small cell lung cancer (NSCLC) is Kirsten rat sarcoma viral oncogene homolog (KRAS)-mutant NSCLC.

19. The method of claim 14, wherein the cancer is colorectal cancer.

20. A method for ameliorating or treating a cancer in a subject in need thereof comprising administering to the subject a therapeutically effective amount of an eIF4A inhibitor and a therapeutically effective amount of a CDK4/6 inhibitor, wherein the eIF4A inhibitor is a compound according to the following formula: 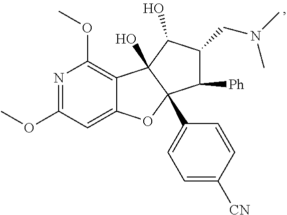
or a stereoisomer, tautomer, or pharmaceutically acceptable salt thereof, and wherein the CDK4/6 inhibitor is selected from the group consisting of palbociclib, ribociclib, and abemaciclib.
